(19) United States
(12) Patent Application Publication
(10) Pub. No.: US 2014/0031605 A1
(43) Pub. Date: Jan. 30, 2014

(54) Method and Use of Peripheral Theta-Burst Stimulation (PTBS) for Improving Motor Impairment

(71) Applicant: Cyril Schneider, Quebec (CA)

(72) Inventor: Cyril Schneider, Quebec (CA)

(73) Assignee: UNIVERSITE LAVAL, Quebec, QC (CA)

(21) Appl. No.: 13/983,177

(22) PCT Filed: Jan. 31, 2012
(86) PCT No.: PCT/CA2012/000079

§ 371 Date: Oct. 14, 2013

Related U.S. Application Data
(60) Provisional application No. 61/438,698, filed on Feb. 2, 2011.

(51) Int. Cl. A61N 2/00

(52) U.S. Cl. CPC A61N 2/008
USPC 600/14

(57) ABSTRACT

The present invention provides new evidences to support that repetitive peripheral magnetic stimulation (theta-burst en stimulation, TBS) over nerve/muscle improves sensorimotor control. Chronic low back pain (CLBP) is associated to a faulty volitional activation of transversus abdominis muscle (TrA) and its delayed contraction during anticipatory postural adjustment (APA), in correlation with maladaptive reorganization of primary motor cortex (M1). Repetitive magnetic stimulation of nerves can influence brain excitability and even reduce rigidity (Parkinson's disease), spasticity (stroke, ABI, cerebral palsy), and contribute to improvement of motor-control and function in stroke, chronic low back pain, ABI, cerebral palsy, prematurity and Parkinson's disease. We hereby test—for the first time—the after-effects of TBS applied over nerves or muscles (peripheral TBS, PTBS) on the motor abdominal-function of chronic low back pain sufferers and on the foot function of brain-injured subjects and to adjust TBS protocol per subject relative to the clinical profile. These pilot studies demonstrate the long-term influence of peripheral neurostimulation in chronic pain, rigidity and spasticity associated to motor impairment.

### CROSS-REFERENCE TO RELATED APPLICATIONS

This application claims priority from U.S. provisional patent application 61/438,698 filed on Feb. 2, 2011, the content of which is incorporated by reference in its entirety.

### FIELD OF THE INVENTION

The present invention relates to a method for improving motor control of a motor impaired subject by applying theta-burst stimulation (TBS) to a peripheral nerve or muscle of the subject. The invention further relates to the novel use of a machine generating electromagnetic signal for the generation of continuous and/or intermittent TBS to a peripheral nerve or a muscle of a motor impaired subject.

### BACKGROUND OF THE INVENTION

Motor Control Impairment in Subjects with Chronic Low Back Pain

Persistence of low back pain (LBP) is associated with lumbar micro-traumatisms (Hodges et al., 2009) due to the impairment of motor control of deep trunk muscles (Hodges and Richardson, 1996; van Dieen et al., 2003) involved in intervertebral control of spine (Bergmark, 1989). Precisely, the transversus abdominis (TrA) presents a faulty volitional control in chronic LBP (CLBP) (Richardson et al., 2004), and, during tasks requiring postural anticipatory adjustments (APA), its activation is delayed (Hodges and Richardson, 1996, 1998) and asynchronous (Massé-Alarie et al., submitted). TrA delay in postural tasks was associated with maladaptive reorganization of the primary motor cortex (M1) (Tsao et al., 2008b). Such reorganization may result from changes in somatosensory cortex (S1) (Flor et al., 1997) and thalamus (Apkarian et al., 2004) due to the influence of sensory inputs on the efficacy of M1 horizontal connections (Kaneko et al., 1994). These cerebral changes may underlie the alteration of proprioception and tactile discrimination in CLBP (Moseley, 2008) and the impairment of sensorimotor control (Richardson et al., 2004). Also, the loss of the short-lasting intracortical inhibition (SICI) of M1 circuits in many chronic pain conditions (Lefaucheur et al., 2006; Mhalla et al., 2010; Schwenkreis et al., 2003) reflects that motor programming mechanisms are altered (Gagne and Schneider, 2008; Ridding and Rothwell, 1999).

Repetitive transcranial magnetic stimulation of M1 that influence corticomotor excitability can decrease pain by reactivating SICI mechanisms in chronic pain (Lefaucheur et al., 2006; Mhalla et al., 2011), however the effects were transient, weak and variable (Maeda et al., 2000). Such limitations could be circumvented by the application of repetitive peripheral magnetic stimulation (RPMS) directly over the nerve/muscle. Indeed, RPMS rather recruits motor fibers than sensory at the periphery (Maccabee et al., 1988), without recruiting nociceptors that could worsen motor impairment (Hodges, 2011). RPMS thus activates the muscle and generates a massive contraction-related flow of proprioceptive information to sensorimotor cortical networks (Struppler et al., 2003). This influence on M1 excitability is meaningful for the control of movement, so that RPMS may improve the ability to activate volitionally a given muscle (Struppler et al., 2003).

Neural plasticity mechanisms triggered by neurostimulation are shared by motor training (Masse-Alarie and Schneider, 2011). Specifically in LBP, motor training of TrA normalized M1 mapping along with improvement of TrA APA delay (Tsao et al., 2010). However, a recent meta-analysis reported that if exercises enhanced function, CLBP was only slightly improved (Hayden et al., 2010). RPMS may be thus used to prime the effects of motor training by opening a therapeutic window when learning becomes easier so that motor control could be better improved and pain reduced.

Therefore, the present study tested, in a single session, whether peripheral neurostimulation over TrA/10 whether alone or in combination with specific motor training improved the outcomes beyond the gains reached by the conventional therapy. Theta-burst stimulation (TBS) protocol was used for the first time as peripheral neurostimulation, because TBS effects on M1 excitability are longer and more consistent than classical protocols of repetitive magnetic stimulation (Huang et al., 2005).

### Muscular Spasticity in Paretic Subjects

A substantial amount of research using several non-invasive stimulation techniques seeks to induce neuroplastic changes within brain' for manipulating the mechanisms underlying deficits in stroke or brain injury² and eventually developing interventional neurostimulation that promotes functional recovery. A twofold challenge in physiopathology is to decrease the debilitating spasticity of anti-gravity muscles (abnormal increase of muscle tone and exaggerated stretch reflexes) that restricts the range of motion at a joint, and to improve in parallel the control/strength of paretic muscles (antagonistic to the spastic). One obvious avenue is to manipulate central nervous system functioning so that hyperactivation of stretch reflexes is abolished. Approaches such as repetitive transcranial magnetic stimulation of the brain can influence cortical excitability at the stimulated site and in remote and functionally networking areas.^{3,4} It was even reported in one study that such neurostimulation over the primary motor cortex (M1) succeeded in decreasing spasticity and improving sensorimotor functions in brain-injured subjects.⁵ However, high-frequency stimulation has to be repeated over several sessions for inducing lasting changes in brain⁶ but with no long lasting changes in function.⁷ Theta burst stimulation (TBS) is an interestingly painless and recently developed variant whose high-frequency bursts of short duration and low intensity induce long-term after-effects that outlast the period of stimulation.⁸⁻¹⁰ Precisely, continuous TBS (cTBS) reduces M1 excitability whereas intermittent (iTBS) increases it⁹, and this likely relies on long-term depression-like and potentiation-like effects.^{11,12} Most TBS studies investigated the basic mechanisms of influence on M1 excitability (as measured by EMG-recorded responses of muscle to M1 stimulation). They also revealed that TBS efficacy (direction, amount and length of after-effects) was dependent on stimulation parameters (intensity, pulse number, inter-stimulus interval, repetitive TBS sessions and inter-session break) and on M1 excitability background (before, during and immediately after stimulation).^{1,10,13-15}

There is thus no consensus yet on TBS protocols that may improve the function in spastic subjects because to date, studies focused on M1 outcomes that depend so closely on brain metaplasticity/homeostatic plasticity.¹³ Whether TBS over M1 can improve spasticity and paresis at a specific joint is unclear though crucial in physiopathology; furthermore, metal-in-jaw/head subjects, e.g. in-metal surgical clipping restoration due to cerebral aneurysm rupture 10 years earlier and up, will not beneficiate from cerebral neurostimulation (see safety guidelines¹⁶). Thus, TBS application at the periphery (PTBS over nerves/muscles) could be a promising alternative that affects sensorimotor control at a specific joint with no limitation in subject's etiology owing to safety guidelines. For example, it was reported that repetitive magnetic stimulation (rPMS) over the innervation zone of the extensor indices proprius muscle increased the excitability at M1 hand representation and improved the reciprocal pattern of activation of wrist flexors and extensors.¹⁷ RPMS over mi-thoracic spinal cord^{18,19} or over lumbar nerve roots^{20,21} decreased spasticity in a broad brain-injured population and even improved function.^{17,22} Thus, proprioceptive inputs generated may not only modulate specific spinal circuits but also influence the cortical plasticity to promote the return of function.

Therefore, we applied first-ever TBS over peripheral nerves (PTBS) to test acute after-effects on spasticity and sensorimotor function of the paretic ankle in chronic brain-injured subjects. First, our PTBS interventional approach was inspired from the baseline knowledge of basic TBS studies in M1: e.g., stimulation was of short duration because after-effects reversal is possible with prolonged stimulation²³ and time-break was imposed between two PTBS sessions for optimal influence². We hypothesized that cTBS over tibial nerve would decrease spasticity of the ankle plantiflexors and iTBS over common peroneal nerve would increase active dorsiflexion, thus promoting the paretic foot function. Also, we proposed-for the first time- to adapt PTBS protocol per subject, i.e. to the actual functional state at baseline and after inter-session break (subject-centered approach). Pre/post-PTBS clinical outcomes denoted dramatic and persistent changes of spasticity and function that are discussed in terms of rapid homeostatic plasticity of brain.

### SUMMARY OF THE INVENTION

The invention therefore provides a method for improving motor control impairment of a subject that comprises applying peripheral neurostimulation.

In a first aspect, the invention therefore provides a method for improving motor control impairment of a subject comprising the step of applying theta-burst stimulation (TBS) to a peripheral nerve or muscle of said subject.

Alternatively, the invention provides the use of theta-burst stimulation to a peripheral nerve or muscle of a subject for improving motor control of a motor impaired muscle of said subject.

In an alternative aspect, the invention provides the use of a machine generating electromagnetic signals for the generation of theta-burst stimulation to a peripheral nerve or muscle of a subject, for improving motor control of a motor control-impaired muscle of said subject.

In an alternative aspect, the invention provides the use of a machine generating electromagnetic signals for the training or treatment of motor-control impairment of a peripheral nerve or muscle of a motor-impaired subject.

In an alternative aspect, the invention provides a method for the treatment of muscular rigidity of a subject suffering therefrom, comprising the steps of: applying theta-burst stimulation (TBS) to a peripheral nerve or muscle of a rigid muscle of said subject.

Alternatively, the invention provides a method for the treatment of muscular spasticity of a subject comprising the steps of: applying theta-burst stimulation (TBS) to a peripheral nerve of a paretic muscle of said subject.

In an alternative aspect, the invention provides the use of intermittent theta-burst stimulation to peripheral nerves of a paretic subject for the treatment of muscular spasticity of peripheral limbs of the subject.

In an alternative aspect, the invention provides the use of a machine generating electromagnetic signal for the generation of intermittent theta-burst stimulation to peripheral nerves of a paretic subject, for the treatment of muscular spasticity of peripheral limbs of the subject.

Other features and advantages of the invention will be apparent from the following Detailed Description, the drawings, and the claims.

### DETAILED DESCRIPTION OF THE INVENTION

### Brief Description of the Drawings

Having thus generally described the aspects of the invention, reference will now be made to the accompanying drawings, showing by way of illustration, particular embodiments thereof, and in which:

FIG. 1. Individual and mean data (+/−SD) of active DF angles)(°) before and after PTBS application in both sessions. S1, S2: sessions 1 and 2; BL: before PTBS in S1 (baseline); pre: before PTBS in S2; post: after PTBS in S1 and S2. **p<0.01. *p<0.05;

FIG. 2. Individual and mean data (+/−SD) of passive DF angles)(°) before and after PTBS application in both sessions. S1, S2: sessions 1 and 2; BL: before PTBS in S1 (baseline); pre: before PTBS in S2; post: after PTBS in S1 and S2. *p<0.05;

FIG. 3. Individual and mean data (+/−SD) of DF catch angles)(°) before and after PTBS application in both sessions. S1, S2: sessions 1 and 2; BL: before PTBS in S1 (baseline); pre: before PTBS in S2; post: after PTBS in S1 and S2. *p<0.05;

FIG. 4. Individual and mean data (+/−SD) of the gap measured between active and passive DF in session 1 (in percentage of passive DF). At BL, the gap is relative to passive DF measured at BL (▾, white bar). At post-PTBS, the gap is relative to passive DF measured at BL (♦, light gray bar) and relative to passive DF measured at post-PTBS (◯, dark gray bar). A gap of 0% denotes that active DF was equal to passive DF. **p<0.01. *p<0.05;

FIG. 5. Summary of the methods with type of stimulation on the target nerves, clinical and neural assessments and outcomes;

FIG. 6. Schematized summary of the stimulating protocols used;

FIG. 7. Examples of mean rectified raw EMG traces of TrA/IO contralateral (cTrA, above graph) and ipsilateral (below graph) to the arm raised during the postural task (N=10 trials per trace in an healthy participant, 100-Hz filtered). Time is expressed relative to AD onset (vertical dotted line, t=0 ms). CTrA/IO onset/end and iTrA/IO onset are arrowed. The shaded area (from 200 ms before to 50 ms after AD onset) corresponds to the time-window when EMG activation is considered as anticipatory for the postural task;

FIG. 8. A. Percentage of trials with anticipatory cTrA/10 activation (upper graph) and with anticipatory iTrA/IO activation (lower graph). B. Onset of cTrA/IO activation (upper graph) and of iTrA/IO activation (lower graph). The horizontal and vertical dotted lines±shaded areas correspond to mean control values±SEM. Pre: pre-stimulation; comb: combination of motor training with either TBS or sham. * p<0,05; ** p<0,01;

FIG. 9. A. Bilateral activation of TrA/IO as calculated by the time elapsed from iTrA/IO onset to cTrA/IO end. The horizontal dotted line±shaded area corresponds to the mean control values±SEM. B. Bilateral activation expressed against the time relative to AD onset (time=0) by time-interval (horizontal lines) between iTrA/IO onset and cTrA/IO onset. The vertical dotted line corresponds to the boundary of anticipatory activation criteria. Pre: pre-stimulation; comb: combination of motor training with either TBS or sham. p<0,05;

FIG. 10. Conditioned MEP of cTrA/IO that reflects the amount of the short lasting intracortical inhibition (SICI). The horizontal dotted line±shaded area corresponds to the mean control values±SEM. Pre: pre-stimulation; comb: combination of motor training with either TBS or sham. * p<0,05; and

FIG. 11. A. Pain scores on visual analogue scale as administrated at pre-stimulation (pre) and post-combination (post-comb). Questionnaire scores for disability (B) and kinesiophobia (C) as administrated at pre-stimulation (pre) and 2 weeks after experiment (post-2w). * p<0,05.

### DEFINITIONS AND ABBREVIATIONS

With respect to the invention presented herein, the following definitions and abbreviations are used, wherein:

ABI: acquired brain injury; BL: baseline; DF: dorsiflexion (aDF, pDF: active dorsiflexion, passive dorsiflexion); TBS: theta-burst stimulation (for example: 5-Hz bursts of 3 pulses delivered at 50 Hz); PTBS: peripheral theta-burst stimulation; cTBS: continuous theta-burst stimulation; iTBS: intermittent theta-burst stimulation, TrA: transversal abdominal muscle; 10: internal oblique muscle; cTrA: contralateral transversal abdominal muscle, iTrA, ipsilateral transversal abdominal muscle; TrA/IO: deep abdominal muscles; SICI: short-lasting intracortical inhibition.

### DESCRIPTION OF PARTICULAR EMBODIMENTS

### Method of Training or Treatment and Use

In accordance with a particular embodiment of this aspect, the invention particularly provides for a method for the treatment of a motor control impairment of a subject suffering therefrom, comprising the steps of: applying intermittent theta-burst stimulation to a peripheral nerve or muscle of a first motor-impaired muscle, or to a peripheral nerve or muscle of an opposite muscle thereof, wherein said first muscle is stimulated to improve motor control thereof.

Particularly, the invention provides the use of a machine generating intermittent theta-burst stimulation to a peripheral nerve or muscle of a first motor-impaired muscle of a subject, for the treatment or training or improvement of muscular motor-impairment.

In an alternative aspect, the invention therefore provides a method for the treatment of muscular spasticity of a subject comprising the steps of: applying theta-burst stimulation (TBS) to peripheral nerves of a paretic muscle of said subject.

In accordance with a particular embodiment of this aspect, the invention particularly provides for a method for the treatment of muscular spasticity of a paretic subject comprising the steps of:
a) intermittently applying theta-burst stimulation to peripheral nerves of a first paretic muscle; and
b) continuously applying theta-burst stimulation to peripheral nerves of a second paretic muscle;
wherein said first and second paretic muscles act in connection with each other for achieving limb movement.

Particularly, the invention provides the use of intermittent theta-burst stimulation to peripheral nerves of a paretic subject for the treatment of muscular spasticity of a peripheral limb of the subject.

Particularly, the invention provides the use of intermittent theta-burst stimulation to peripheral nerves of a first paretic muscle of a subject, and continuous theta-burst stimulation to peripheral nerves of a second paretic muscle of said subject for the treatment of muscular spasticity, wherein said first and second paretic muscles act in concert for achieving limb movement.

In a third aspect, the invention provides the use of a machine generating electromagnetic signal for the generation of intermittent theta-burst stimulation to peripheral nerves of a paretic subject, for the treatment of muscular spasticity, of peripheral limbs of the subject.

Particularly, the invention provides the use of a machine generating:
a) intermittent theta-burst stimulation to peripheral nerves of a first paretic muscle of a subject, and
b) generating continuous theta-burst stimulation to peripheral nerves of a second paretic muscle of said subject,
   for the treatment of muscular spasticity, wherein said first and second paretic muscles act in concert for achieving limb movement.

### Medical Indications

Particularly, as it relates to the above-mentioned aspects, the invention applies to a premature baby, or a subject having chronic low back pain, peripheral muscle rigidity or peripheral muscle spasticity.

More particularly, according to the different aspects of the invention, the subject having peripheral muscle rigidity is a subject having Parkinson's disease.

More particularly, the subject is paretic and suffers neuronal loss because of, for example, stroke, aneurysm rupture, cerebral palsy or acquired brain injury (ABI).

### Subjects

In accordance with the invention's different aspects, the subject is a mammal. Particularly, the subject is a human subject.

Particularly, the subject is submitted to peripheral theta-burst stimulation (PTBS) at one or more than one peripheral nerve or one or more than one muscle. More particularly, peripheral theta-burst stimulation is applied is applied as intermittent TBS on an underactivated muscle. Still, most particularly, intermittent TBS consists of a 2-sec train of PTBS repeated every 10 sec for a total of 190 sec (600 pulses).

Alternatively, the PTBS is applied as continuous TBS on a hyperactivated muscle. Still, most particularly, continuous TBS consists of a 40-sec train of uninterrupted PTBS (600 pulses).

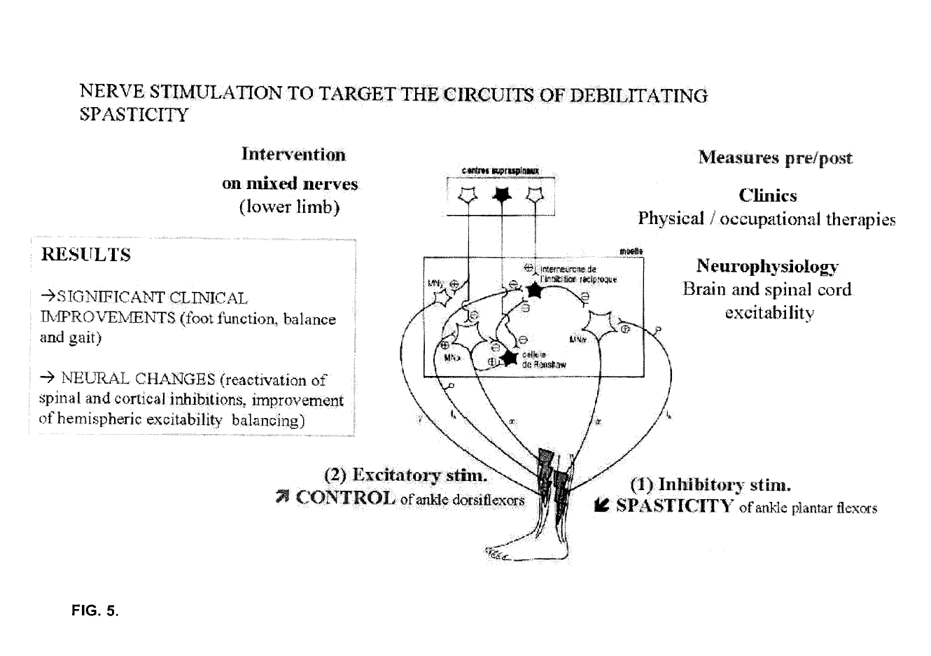

Still more particularly, peripheral theta-burst stimulation is applied to a first paretic muscle and a second paretic muscle sequentially. More particularly, the PTBS is applied sequentially as continuous TBS on a first paretic hyperactivated muscle and as intermittent TBS on a second paretic underactivated muscle. Still most particularly, the first paretic muscle is stimulated by cTBS to decrease spasticity of the first muscle and the second paretic muscle is stimulated by iTBS to increase contraction of the second muscle. (FIG. 5).

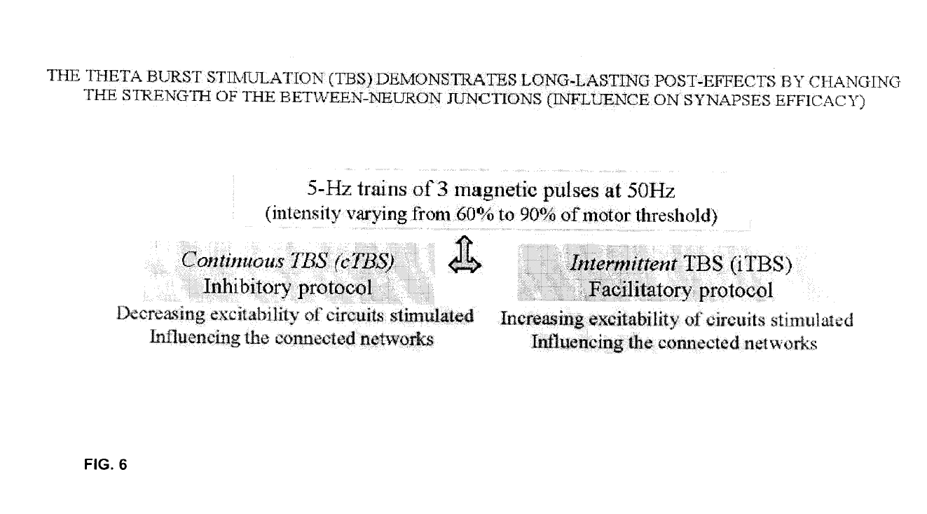

Most particularly, PTBS are delivered with 5-Hz bursts (for example each 200 ms) of three pulses at 50 Hz (for example each 20 ms) (FIG. 6). Still, even most particularly, cTBS₆₀₀ consists of a 40-sec train of uninterrupted PTBS (600 pulses) and iTBS₆₀₀ of a 2-sec train of PTBS repeated every 10 sec for a total of 190 sec (600 pulses).

The following examples are intended to illustrate, rather than limit, the invention.

### Example 1

### The Use of Peripheral Theta Burst Stimulation to Reinstall Foot Function in Spastic Brain-Injured Subjects

### Materials and Methods

### Subjects

Six subjects aged 39.3 years (SD=16.5, range 20-57) were enrolled in this study under written informed consent approved by local ethics committees. Subjects had chronic stroke (2 men), aneurysm rupture (1 man, 1 woman with anti-convulsive medication), and severe acquired brain injury (2 women) (see Table 1). The birth handedness was determined according to Oldfield's inventory²⁴. The inclusion criteria were spasticity at baseline (BL) for the plantiflexors of the paretic ankle and deficits in active dorsiflexion (DF), i.e. non functional-for-walking active DF (≦0°). A range of motion (ROM) of 0° DF represents a 90°-angle between foot and leg, as measured under standardized procedure. Negative vs. positive ROM (or less negative) represent reduced vs. increased DF.

TABLE 1
Patients' characteristics.

| | | | | | Stimulated |
|---|---|---|---|---|---|
| | | | | | ankle & |
| | | Age | Time since | Nature and location | contralateral |
| Patient | Sexe | (yrs) | lesion (yrs) | of lesion | H |
| P1 | F | 54 | 31 | Right sylvian | Left, NDH |
| | | | | aneurysm | |
| P2 | M | 50 | 15 | Right fronto- | Left, NDH |
| | | | | temporal | |
| | | | | aneurysm | |
| P3 | M | 33 | 4 | Left ischemic | Right, DH |
| | | | | stroke: | |
| | | | | perisylvian, | |
| | | | | brain stem, | |
| | | | | hemicerebellum | |
| P4 | M | 57 | 6 | Left ischemic | Right, DH |
| | | | | stroke: fronto- | |
| | | | | temporo- | |
| | | | | occipital | |
| P5 | F | 20 | 2 | Severe ABI | Right, DH |
| P6 | F | 22 | 2 | Severe ABI | Right, NDH |

yrs: years;
F, M: female, male;
ABI: acquired brain injury;
H: hemisphere;
D, ND: dominant, non dominant (as refereed to handedness).

### Clinical Measures

A research therapist assessed the active (volitional) and passive (manually imposed) DF (paretic aDF, pDF) with an extendable manual goniometer (Lafayette Instrument Cie, ±5° measurement error, ±8° changes for clinical significance²⁵). Two successive aDF were measured, the best was retained; one pDF was measured to avoid repetitive muscle stretching. Ankle spasticity was evaluated once by the ‘catch’ angle at which pDF was blocked by the hyperactive stretch reflex of plantiflexors. Given that spasticity is velocity-dependent²⁶, 1-sec high-speed DF stretching was used by having the therapist count silently (<<one-thousand-one>>) so that, e.g., a 30-degree pDF was imposed at 30°/sec (reproducible methods²⁷). ADF then pDF were always measured before spasticity for avoiding data contamination by stretch reflex hyperactivation.

### PTBS Technique

CTBS and iTBS were used in combination, with cTBS applied at first to decrease spasticity of the ankle plantiflexors (Triceps surae) and iTBS applied immediately after to promote the control of the ankle dorsiflexors (e.g. Tibialis anterior) and eversors. To this end, cTBS was applied over the tibial nerve (TN, located centrally in the popliteal fossa) and iTBS over the common peroneal nerve (CPN, directly posterior to the head of fibula).

Stimuli were delivered through a high-frequency magnetic stimulator (Magstim Rapid²; The Magstim Company Ltd, Whitland, UK) connected to an air film cooled coil (figure-of-eight, 7-cm mean loop diameter, magnetic stimulus with biphasic waveform and a pulse width of 400 ms). The coil was held tangentially to the skin over the nerve spot with the coil heading upwards at 45-deg from the nerve direction, this coil orientation being most effective for biphasic stimulation.^{4,10} Stimulus intensity (90% of motor threshold) was set so that PTBS induced palpable muscle contraction and visible movement (plantiflexion vs. dorsiflexion/eversion): therefore, M-waves in alpha motoneurons ensured that peripheral stimulation effectively recruited most sensory-afferent myelinated fibers (with larger diameter thus lower membrane resistance). Care was taken to ensure that muscle contraction and the PTBS-induced sensation reported by each subject corresponded to the innervation patterns of the same nerve, then skin marquees ensured a reliable coil positioning. PTBS were delivered with 5-Hz bursts (each 200 ms) of three pulses at 50 Hz (each 20 ms).⁹ Precisely, cTBS₆₀₀ consisted of a 40-sec train of uninterrupted PTBS (600 pulses) and iTBS₆₀₀ of a 2-sec train of PTBS repeated every 10 sec for a total of 190 sec (600 pulses).

### Experimental Design

Each subject participated to 2 sessions (S1, S2) where aDF, pDF and spasticity measures were strictly repeated at pre- and post-PTBS. An S1-S2 time-break (7 days at least) was imposed for having the second stimulation session still induce facilitatory after-effects.² Post-PTBS measures were done at 10 min after PTBS completion, given that cortical TBS effects are maximal at 10-30 min post-stimulation” and that the inhibitory after-effects of cTBS can be reversed to facilitation if muscle contraction immediately follows cTBS¹³. The subject was comfortably seated in a reclining and adjustable chair, the arms relaxed on adjustable supports, and the knees at 25° flexion to prevent from excessive stretching of the spastic ankle plantiflexors.

### Within-Subject Adjustment of PTBS Protocol

PTBS protocol was adjusted in each session according to pre-PTBS spasticity and foot function in this open-label pilot study (see Table 2). At BL, no cTBS was elicited when aDF was greater than the catch angle (i.e. aDF was not obstructed by spasticity) and when pDF was close to 0° (±5° measurement error), so that a functional-to-walking ROM could be obtained in aDF. At S2, PTBS protocol was adjusted relative to data obtained in S1. Table 3 shows that PTBS could promote pDF in S1, thus pDF measurement may include spastic components (see Example 3). We therefore considered that, at S2 (pre-PTBS_{S2}), if DF catch angle or/and pDF was decreased by more than 5° as compared to S1 (post-PTBS_{S1}), this would reflect an offline increase of spasticity. In this case, cTBS was given at S2 (subjects P3, P5, P6); else, cTBS was not repeated at S2 to avoid reversal of after-effects when improvements were maintained.^{13,14} It is noteworthy that P2 presented important spasticity and paresis at the knee joint, thus cTBS was also applied over the femoral and sciatic nerves.

TABLE 2
PTBS protocols in S1 and S2 in each patient.

| Patient | PTBS at S1 | PTBS at S2 | S1-S2 break (days) |
|---|---|---|---|
| P1 | no cTBS | no cTBS | 7 |
| | iTBS on CPN | iTBS on CPN | |
| P2 | cTBS on TN | no cTBS | 7 |
| | iTBS on CPN | iTBS on CPN | |
| | | (and iTBS on FN, SN) | |
| P3 | cTBS on TN | cTBS on TN | 35 |
| | iTBS on CPN | iTBS on CPN | |
| P4 | cTBS on TN | no cTBS | 7 |
| | no iTBS | iTBS on CPN | |
| P5 | no cTBS | cTBS on TN | 14 |
| | iTBS on CPN | iTBS on CPN | |
| P6 | cTBS on TN | cTBS on TN | 21 |
| | iTBS on CPN | iTBS on CPN | |

S1, S2: sessions 1 and 2;
iTBS, cTBS: intermittent (190 sec) and continuous (40 sec) theta-burst stimulation (600 pulses each);
CPN: common peroneal nerve;
TN; tibial nerve;
FN: femoral nerve;
SN: sciatic nerve.

PTBS protocol adjustment was different in P1 and P4 because no aDF was present at BL (see Table 3). No cTBS was applied for P1 with positive pDF angles in S1 and S2. Only cTBS was applied for P4 in S1 due to very small passive movements possible; cTBS was switched to iTBS in S2 due to the maintained improvements of DF catch angle and pDF.

### Statistics

Repeated measure analyses of variance (ANOVA_{RM}) with TIME of measurements (4 levels) and SIDE stimulated (2 levels) as within subject factors were applied on aDF, pDF and DF catch angles. TIME included the pre/post-PTBS measures in S1 and S2 as respectively refereed to baseline (BL), post-PTBS_{S1}, pre-PTBS_{S2} and post-PTBS_{S2}. SIDE denoted the paretic side and its contralateral injured hemisphere (Dominant vs. Non Dominant relative to birth handedness) that received sensory information generated by PTBS. Pearson's correlation test was used to examine correlation between TIME. Subjects P1 and P4 with no movement at BL were withdrawn from aDF group means and ANOVA_{RM} and their active data treated separately.

The gap [Active—Passive] DF (Δ) was calculated only in S1 to assess PTBS acute effects on the active performance relative to ankle stiffness. ΔBL was calculated as follows: [Active_{BL}−Passive_{BL}]*100/Passive DF_{BL}. Due to denoted changes in passive DF, Δpost-PTBS was expressed first relative to passive DF at BL (Δpost/BL): [Active_{post-PTBS}−Passive_{BL}]*100/Passive_{BL}, then relative to passive DF at post-PTBS (Δpost/post): [Active_{post-PTBS}−Passive_{post-PTBS}]*100/Passive_{post-PTBS}. Differences between ΔBL, Δpost/BL, and Δpost/post were tested independently using an ANOVA_{RM} with TIME (4 levels) as factor. Age, sex, nature and time from lesion, and S1-S2 time break were not considered factors for ANOVA_{RM} due to the small sample. Statistical power (SP) was calculated from Cohen's d-algorithm.²⁸ Level of significance was set at p<0.05.

### RESULTS

ANOVA_{RM} detected the only main effect of TIME for all variables thus no post-hoc comparisons was required. Pearson's correlation between all TIME was very strong (r=0.83 to 0.98 depending on the variable) and reflects the reproducible changes between subjects who did not report any side effects in S1 or S2.

TABLE 3
Individual and mean data for active,
passive and catch DF angles.

| | | | | |
|---|---|---|---|---|
| | | post- | pre- | post- |
| Patient | BL | PTBSₛ₁ | PTBSₛ₂ | PTBSₛ₂ |

Active DF (°)

| | | | | |
|---|---|---|---|---|
| P1* | No mvt | −10 | −30 | −10 |
| P2 | −20 | −5 | −14 | −3 |
| P3 | 0 | 12 | 8 | 13 |
| P4* | No mvt | No mvt | No mvt | No mvt |
| P5 | −20 | −7 | −10 | −2 |
| P6 | −20 | 0 | −20 | −5 |
| Mean | −15.0 | 0.0 | −9.0 | 0.8 |
| SD | 10.0 | 8.5 | 12.1 | 8.3 |

Passive DF (°)

| | | | | |
|---|---|---|---|---|
| P1 | 10 | 3 | 7 | 7 |
| P2 | −10 | 0 | 5 | 7 |
| P3 | 18 | 20 | 12 | 15 |
| P4 | −35 | −15 | −20 | −13 |
| P5 | −4 | −2 | −8 | 5 |
| P6 | −10 | 10 | 0 | 5 |
| Mean | −5.2 | 2.7 | −0.7 | 4.3 |
| SD | 18.5 | 11.8 | 11.7 | 9.3 |

Catch angle (°)

| | | | | |
|---|---|---|---|---|
| P1 | −10 | −3 | −13 | −10 |
| P2 | −21 | −15 | −6 | −3 |
| P3 | −20 | −20 | −21 | −16 |
| P4 | −50 | −35 | −32 | −25 |
| P5 | −12 | −25 | −17 | −15 |
| P6 | −28 | −11 | −10 | −10 |
| Mean | −23.5 | −18.2 | −16.5 | −13.2 |
| SD | 14.5 | 11.2 | 9.2 | 7.4 |

DF: dorsiflexion;
BL: baseline;
S: session;
pre, post: before and 10′ after PTBS;
mvt: movement;
SD: standard deviation.
*In active DF, P1 and P4 (shadowed) were not included in group means and ANOVA_{RM}.

### Active DF

Subjects P1 and P4 with no aDF at BL were withdrawn from aDF group means and ANOVA_{RM} (see Table 3). ITBS reinstalled aDF in P1: it was triggered at S1)(−10°, somewhat maintained until S2 (−30°) and increased again in S2 (−10°), along with stable pDF and DF catch angles. In P4, cTBS and iTBS did not enable any aDF at S1 and S2, respectively, but pDF and DF catch angles were 50% dramatically increased.

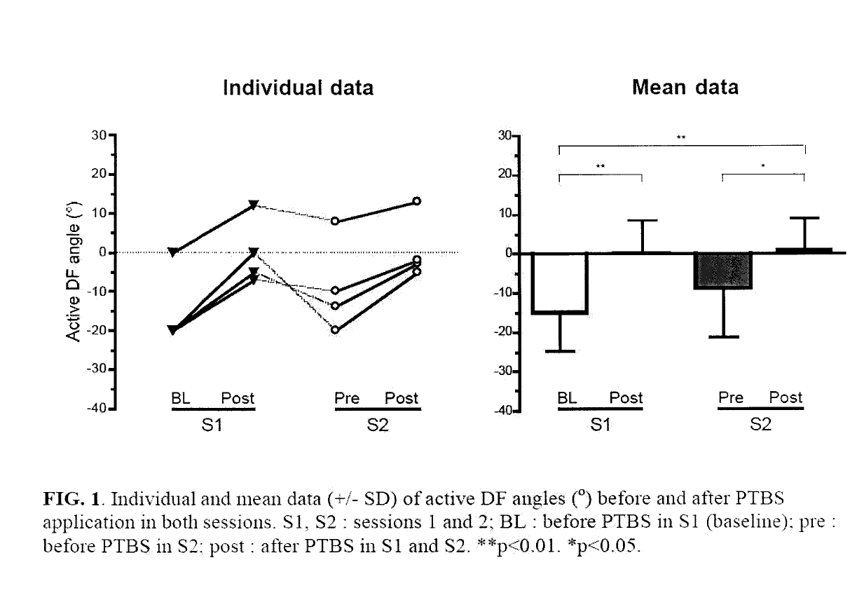

ADF was significantly increased for the other 4 subjects in post-PTBSₛ₁ as compared to BL (F_{1,2}=138.5, p=0.007; SP=86.36%; FIG. 1). Values decreased at pre-PTBS_{S2} but with no significant-level difference with post-PTBS_{S1} (F_{1,2}=10.6, p=0.083). ADF still increased in post-PTBS_{S2} as compared to pre-PTBS_{S2} (F_{1,2}=60.8, p=0.016; SP=53.6%) and overall, it was far better in post-PTBS_{S2} as compared to BL (F_{1,2}=136.9, p=0.007; SP=91%).

### Spasticity and Passive DF

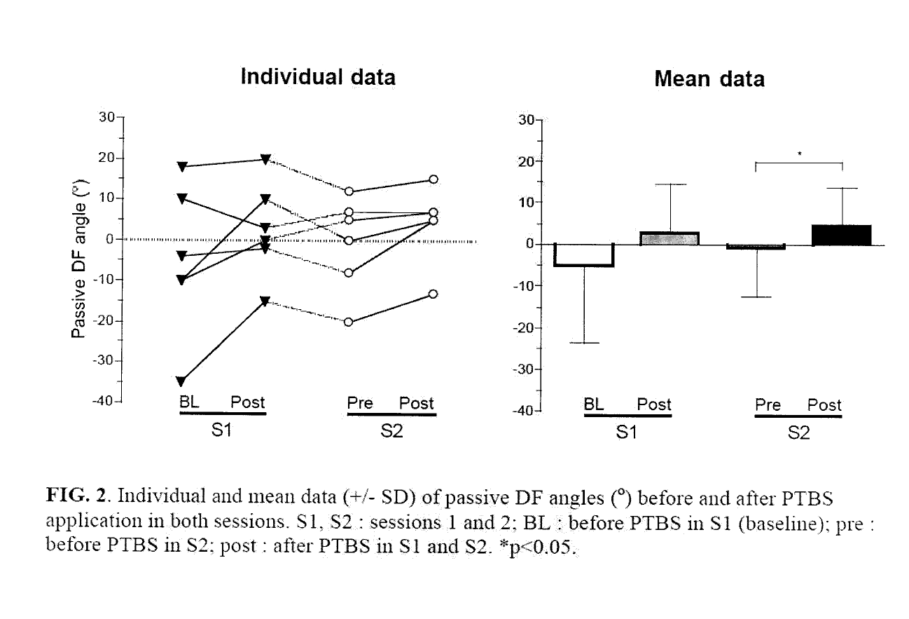

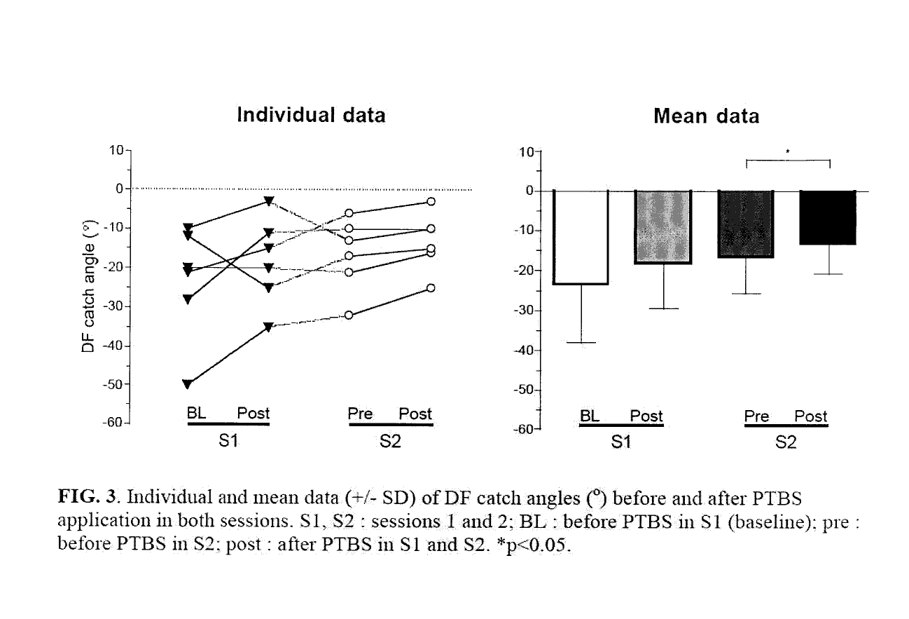

DF catch angle and pDF were improved post-PTBS but the increase was significant only at S2 although this was expected only for DF catch angles. Precisely, pDF was greater in post- than in pre-PTBSₛ₂ (F_{1,4}=9.5, p=0.037; SP=84%; FIG. 2) and the same for DF catch angles (F_{1,4}=14.3, p=0.019; SP=70%; FIG. 3).

### Active DF in S1 Relative to pDF

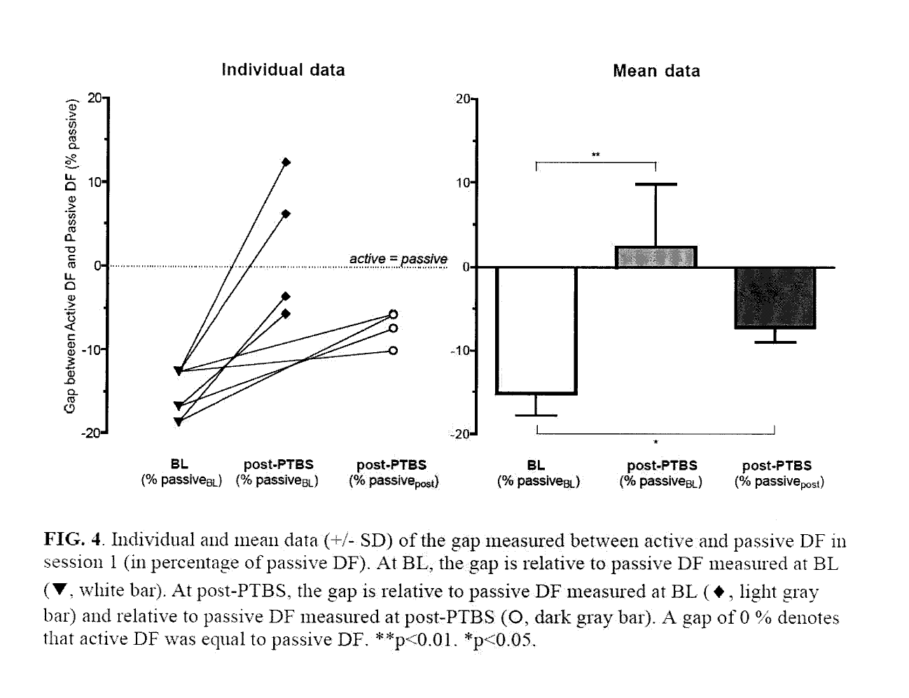

The gap Δpost/BL (mean=2.4%, SD=8.5%) between aDF_{post-PTBS} and pDF_{BL} was reduced by 17.5% (SD=6%) as compared to the gap ΔBL (mean=−15.08%, SD=3%) between aDF_{BL} and pDF_{BL} (F_{1,3}=35.1, p=0.009; SP=94.6%; FIG. 4-center plot). Δpost/BL became even positive (aDF greater than pDF) which would have been impossible if pDF had remained stable after PTBS. This suggests acute PTBS effects on pDF in S1, as in S2 (previous section). The gap Δpost/post (mean=−7.1°, SD=2° between aDF_{post-PTBS} and pDF_{post-PTBS} was also significantly reduced as compared to BL gap (mean reduction of 7.94%, SD=4.4%, F_{1,3}=13.16, p=0.036; SP=99.93° A; FIG. 4-right plot).

### Discussion

This pilot study in 6 brain-injury cases is the first study on peripheral TBS (PTBS) and on adaptation of PTBS protocol to each subject's clinical profile. The original findings indicate significant improvements in foot function, as denoted by acute and long-term decrease of ankle plantiflexors spasticity and increase of volitional aDF.

### Methodological Considerations and Clinical Significance

The subject-centered adjustment of PTBS protocol provided consistent group data (e.g., mean raw aDF increase of 15° in both sessions for all subjects) that were statistically and clinically significant (above 8° changes²⁵, see FIGS. 1B-4B), whatever the nature of brain lesion, age, sex, dominance of side stimulated, time post-lesion, and S1/S2 time-break. Consistent group data suggest that, even if PTBS was very brief, it was not more susceptible to individual differences than longer lasting methods. Sham-controlled and double-blinded experiments will be designed in larger-sample studies. They were not considered here since PTBS-induced changes were immediate and dramatic in subjects clinically stable for years, with no influence of conventional rehabilitation and no physical therapy at time of experiments. Data denoted also that the acute changes at first PTBS application could be maintained offline during S1-S2 break (no significant alteration or suppression), and still recovered at second PTBS application within the boundaries of passive ROM. Since no stretching intervention was administrated, the significant increases of pDF at S1 and S2 (see FIG. 4B-2B, respectively) suggest that viscoelastic passive measures might have been contaminated by muscle spasticity decrease though methodological care. The linear decrease of spasticity in most subjects over time confirms rPMS efficacy on spasticity²⁹ and encourages TBS use as an anti-spastic adjuvant in post-stroke rehabilitation to improve motor control.

### Possible Underlying Mechanisms

The respective cTBS/iTBS after-effects were not compared together but we actually combined them at the periphery on the basis of what was acknowledged about 600-pulse design over brain. TBS is a rapid method of producing long lasting after-effects on the excitability of the stimulated M1. Could PTBS over the nerves produce the same homeostatic plasticity that modifies motor control?¹ TBS protocols of M1 to improve function are based on the imbalancing of interhemispheric inhibitory interactions between ipsi- and contra-lesional M1 after brain injure when the damaged hemisphere is not only disabled by neuronal loss, but also by increased interhemispheric inhibition from the contralesional hemisphere³¹. Thus TBS that modulates cortical excitability can <<rebalance>> inter-hemispheric interactions and normalize the cortical excitability of both the injured and non-injured hemispheres. In the same vein, PTBS may activate proprioceptive signals that generate movement-like activity at the somesthetic level via lemniscal pathways and influence M1 excitability via corticocortical fibers^{17,32} and contralateral M1 via transcallosal or sub-callosal routes³¹. PTBS-generated sensory feedback may thus improve motor planning. For example, together with corticospinal drive for ankle dorsiflexion, PTBS may reactivate the inhibitory descending controls on the spinal circuits of plantiflexors to prevent from stretch reflex. That is, whether cTBS of tibial nerve reactivated the lacking la-presynaptic inhibition in spastic subjects³³ and iTBS of common peroneal nerve normalized M1 excitability and/or reduced spasticity too³⁴, should be addressed in futures studies measuring PTBS after-effects on neural excitability at the spinal and cortical levels. ADF improvement in most subjects while spasticity could worsen supports that neuroplastic changes differed between aDF increase (likely at cortical level for motor planning) and reduction of spasticity (spinal reflex hyperexcitability tested at rest) that required more time likely for installation of cortical changes at the spinal level³³.

### Short-Term Perspectives in Neuro-Rehabilitation

This original pilot study presents that PTBS results in sustained improvement of spasticity and function years after stroke, aneurysm rupture or ABI (in one case, dorsiflexion was triggered 30 years after no ankle movement). PTBS is thus a new easy-to-administer adjuvant in neuro-rehabilitation for chronic subjects living with spasticity. Carry-over to more complex tasks such as mobility should be tested in future studies as well as underlying neural changes and their dependence on stimulation parameters, brain metaplasticity and subjects' characteristics. Overall, PTBS provides a therapeutic window (length of after-effects) during which brain responsiveness to training may foster the cortical reorganization that enables improvement beyond the functional gains already reached in conventional therapy³⁵.

### Example 2

### Peripheral Neurostimulation and Specific Motor Training of Deep Abdominal Muscles Improve Motor Control of Postural Adjustment in Chronic Low Back Pain Subjects

### Methods

### Participants and Study Design

Thirteen right-handed individuals with CLBP (≦1-year pain, mean age=53.7±7.4 years, ranging 37-61 years, 6 males, Table 4) were recruited for one single session from our local Pain Management Centre and Physiotherapy Unit under informed written consent approved by local ethics committees, conform to the Helsinki Declaration. Nine healthy right-handed individuals (mean age=48.7±6.8 years, ranging 36-55 years, 4 males, Table 4) with no LBP affection in the last year were included as control group. This study was double-blind, randomized, placebo and controlled. Subjects were randomly allocated to 2 groups: 7 received peripheral TBS (TBS group: TBS alone then TBS+motor training), 6 received sham stimulation (Sham group: Sham alone then Sham+motor training). The exclusion criteria were LBP induced by fracture, malignancy, more than 2 radicular signs, lumbar infiltrations (≦6 months before enrollment), facet denervation, lumbar surgery other than laparoscopy, other chronic pain pathology, litigation, any form of abdominal training (≦1 year), any major circulatory, respiratory, neurological or cardiac diseases, severe orthopedic troubles, cognitive deficit, infection or recent/current pregnancy. Exclusion criteria related to TMS testing are reported elsewhere (Rossi et al., 2009) and mainly concerned brain surgery, lesion or injury, any history of seizure or concussion, pacemaker/pump holder, change of medication (≦2 weeks preceding enrollment), metallic implants in skull or jaw. CLBP subjects were tested for TrA motor patterns during a postural task and for TrA M1 excitability (TMS testing) at 3 different times of measurement, i.e. before peripheral stimulation (TBS or sham), after stimulation alone and after [stimulation+motor training] combination. The physiotherapist performing clinical evaluation and motor training was blinded to group allocation. Healthy participants were tested once for the same outcomes.

TABLE 4
Descriptive statistics of 3 groups (means ± SD)

| | Control | TBS group | Sham group | |
|---|---|---|---|---|
| | (n = 9) | (n = 7) | (n = 6) | p |
| Age (years) | 48.7 ± 6.8 | 53.6 ± 5.5 | 53.8 ± 9.8 | 0.96 |
| Gender | 5/4 | 4/3 | 2/4 | |
| (Females/Males) | | | | |
| Height (m) | 1.68 ± 0.08 | 1.65 ± 0.11 | 1.59 ± 0.05 | 0.24 |
| Weight (kg) | 69.8 ± 10.7 | 84.8 ± 21.1 | 72.8 ± 12.7 | 0.24 |
| Pain VAS (/10) | | 3.4 ± 2.5 | 2.5 ± 2.6 | 0.54 |
| Pain duration (years) | | 19.0 ± 11.3 | 12.6 ± 7.7 | 0.25 |
| QBP | | 30.6 ± 15.9 | 31.5 ± 21.4 | 0.93 |
| TSK | | 41.4 ± 9.3 | 40.3 ± 6.0 | 0.80 |

VAS: visual analog scale;
QBP: Quebec back pain disability scale;
TSK: Tampa scale of kinesiophobia.
p: 2-tailed unpaired t-test

### Clinical Evaluation and Questionnaires

Pain and spine motor control were conventionally evaluated in all CLBP subjects by an expert physiotherapist. Prone instability test, aberrant lumbar motion and passive straight leg raising were assessed because they are predictive indicators of a successful lumbar stabilization training (Hicks et al., 2005) and thus are integral to diagnosis. CLBP subjects also self-assessed their pain before and after testing (Visual Analogue Scale for pain) and fulfilled 2 more questionnaires before and 2 weeks after testing (Tampa Scale for Kinesiophobia', TSK, (French et al., 2002), and ‘Quebec Back Pain Disability Scale’, QBP, (Kopec et al., 1995)).

### Surface EMG Recordings

Conversely to fine-wired intramuscular electrodes, surface EMG recordings cannot discriminate between TrA and 10 activation. The acronym TrA/10 is thus used. EMG activity was collected using surface parallel-bar EMG sensors positioned with adhesive skin interfaces over the TrA/10 bilaterally so that cTrA/10 and iTrA/10 activity could be recorded, and over EO and anterior deltoid (AD) muscles unilaterally (Ng et al., 2002); reference ground was positioned on the iliac crest (16-Channel Bagnoli EMG System, Delsys Inc., Boston, Mass.). TrA/10 electrodes were placed 2 cm inferior and 2 cm medial to antero-superior iliac spine (Marshall and Murphy, 2003) so that the most superficial position of the muscle (TrA and inferior 10) was reliably recorded (Marshall and Murphy, 2003), and a study using such electrodes placement has reported TrA and 10 activation delay in LBP subjects during rapid arm raising (Hodges and Richardson, 1999). EMG signals were bandpass-filtered (20 Hz-450 Hz), amplified before digitization (2 kHz), and computer-stored for online display and offline analysis (PowerLab acquisition system, LabChart-ADlnstruments, Colorado Springs, Colo.). During TMS testing (see next section), real-time EMG activity of cTrA/10 (contralateral to arm raised) was monitored to ensure that participants maintained background EMG at 15% of maximal voluntary contraction (MVC). EMG activity of cTrA/10 was 2-Hz filtered and synchronized on a computer screen where participants had to match the 15% MVC target displayed as a line. MVC was determined at onset of experiment by the mean cTrA EMG activation obtained from 3 maximal expirations of 3 seconds each.

### TMS Testing

Participants were comfortably seated in a reclining and adjustable chair with arm supports and knees extended and TMS was applied while they maintained the cTrA/10 preactivated at 15% of MVC using a visual biofeedback. Precisely, magnetic stimuli were applied over M1 abdomen territory (10-20 EEG localization) using a double-cone coil (7-cm outer diameter each wing) connected to two Magstim 200² monophasic stimulators (BiStim² module, The Magstim Company Limited, Whitland, UK). Double-cone coil was used because consistent motor evoked potentials (MEP) of TrA/10 were recorded during preliminary testing at minimal intensities compared to figure-of-eight coil. The double-cone coil midline center was positioned 2 cm-lateral and 2 cm-anterior to Cz (10-20 EEG system) at an angle of 45° to the sagittal plane for inducing current in the antero-medial direction (Sakai et al., 1997), so that consistent MEP of contralateral TrA/10 could be obtained at lowest TMS intensities and for slight muscle contraction (Strutton et al., 2004; Tsao et al., 2008a). This usual abdominal muscles hotspot in M1 could be shifted postero-laterally in LBP, as already reported (Tsao et al., 2008b). Both hemispheres were stimulated to determine the ‘dominant’ side to stimulate solely during the experiment, i.e. the hemisphere with the largest cTrA MEP at lowest intensity. This M1 hotspot was marked with an oily-tipped pen on the scalp and this served as a visual reference for reliable coil positioning. The active motor threshold (AMT) was determined as the TMS intensity eliciting at least 5 MEP equal to or greater than 100 μV out of 10 trials with cTrA/10 at 15% MVC. SICI was tested using the paired-pulse TMS paradigm (Kujirai et al., 1993) shown as measuring inhibition of pure cortical origin in preactivated conditions (Ortu et al., 2008). Precisely, the subthreshold conditioning stimulus (70% AMT) was delivered 2 ms before the suprathreshold test stimulus (120% AMT) and 8-10 test (unconditioned) MEPs and 8-10 conditioned MEPs were recorded (Ortu et al., 2008). Trials falling outside a stringent window of EMG acceptance were rejected online (15% MVC+/−5%).

### Postural Task

Participants performed 10 rapid shoulder flexions while quiet standing, this number of trials being reported reliable in such a task (Tsao et al., 2010). This created internal perturbations to the trunk and requiring postural adjustments. The arm raised was contralateral to the ‘dominant’ cTrA/10 (as determined in previous TMS section) and was not necessarily the dominant arm. Precisely, shoulder with full-extended elbow had to be flexed to 90° as fast as possible from the relaxed vertical position along the trunk in reaction to an auditory tone. Practice trials were scheduled. Any trial assessed visually as not performed at maximal velocity was discarded and repeated until 10 trials recorded.

### Peripheral TBS and Sham

CLBP subjects were positioned in dorsal decubitus with TrA/10 relaxed (monitored online by visual feedback of EMG background). TBS group received intermittent TBS over the cTrA/10 spot the most accessible, i.e. 2 cm medial and inferior to antero-superior iliac spine. This protocol corresponds to bursts of 3 magnetic pulses at 50 Hz, repeated at 200-ms intervals (5 Hz) for 2s, followed by 8s OFF (Huang et al., 2005). This protocol was repeated 3 times (10-minutes total). The intensity was set at 33% of maximal stimulator output (MSO), generating palpable TrA contraction. Sham group also received TBS (same protocol) but at very weak intensity (5% MSO). Peripheral magnetic stimulation recruits preferentially motor fibers (Maccabee et al., 1988), thus weak intensity with no overt muscle contraction may have not generated proprioceptive flow to S1 (Zhu and Starr, 1991).

### Motor Training

The physiotherapist supervised CLBP subjects for TrA motor training. Precisely, subjects were in a crook-lying-position and had to gently hollow (draw-in) lower abdomen while keeping a normal respiratory cycle (Urquhart et al., 2005). They had to maintain TrA/10 contraction at 15% MVC following EMG biofeedback (2-Hz filtered) displayed on a computer screen. Contraction of 15% MVC was convenient since a low-intensity training mimics functional activation of TrA (Tsao et al., 2008b). Once subjects were able to isolate TrA contraction (maximal reduction of other abdominal muscle activity as monitored by online surface EMG recordings), 3 sets of 10 hollowing repetitions (10s each) were performed at 2-minutes intervals (Tsao and Hodges, 2007).

### Data Reduction and Statistical Analysis

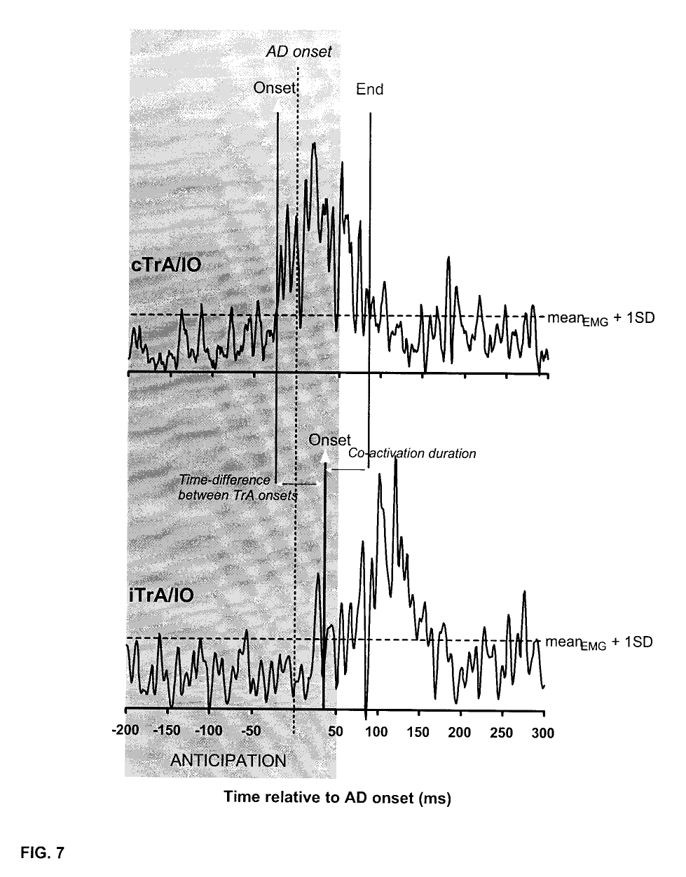

Three TMS outcomes were studied per participant for cTrA/10: AMT (M1 basic excitability); mean peak-to-peak amplitude of test MEPs; mean peak-to-peak amplitude of conditioned MEPs (% of test MEP amplitude, (Di Lazzaro et al., 1998). Five outcomes were calculated during APA of the postural task (see FIG. 1): the respective onsets of cTrA/IO and iTrA/IO bursts (ms) were expressed relative to AD onset (primum movens of shoulder flexion); the time-interval (ms) between iTrNIO onset and the end of cTrA/IO burst tested the bilateral activation; the number of trials with anticipatory activation of cTrA/IO and the number of trials with anticipatory activation of iTrA/IO (i.e. from 200 ms before to 50 ms after AD activation) were expressed in percentage of the 10 trials. All previous time measures were detected visually by the increase of EMG background above the baseline corresponding to quiet standing. This method was reported reliable and not dependent on the baseline activity (Hodges and Bui, 1996). Precisely, TrA/10 EMG signals were full-wave rectified, 50-Hz filtered (RMS technique), and the EMG background was averaged during the first 200 ms (before the auditory cue) to determine the baseline activity in each trial (FIG. 7). Muscle activation was detected by EMG activity above one standard deviation from the baseline and lasting at least 50 ms (TrA/10 or AD, (Hodges and Bui, 1996). This activation was defined as anticipatory when it occurred from 200 ms before to 50 ms after AD onset (Aruin and Latash, 1995).

A two-way ANOVA was applied on TMS outcomes of cTrA/IO and on APA variables (cTrA/IO and iTrA/IO independently) using factors GROUP (TBS vs. Sham)×TIME of measurement (Pre-, Post-stimulation, Post-combination). A two-way ANOVA was applied on pain/function scores (from functional questionnaires) using factors GROUP (TBS vs. Sham)×TIME of measurement for VAS (Pre-experiment vs. Post-combination) and for questionnaires scores (Pre-experiment vs. 2 weeks post-experiment). ANOVAs were conducted with repeated measures on TIME (ANOVA_{RM}). Planned comparisons tested where differences lay. Relations between the different outcomes were tested with Pearson coefficient. The level of significance was set at p<0.05. A one-way ANOVA with factor Group (TBS, Sham, Control), repeated at the 3 times of measurement, was performed on variables significantly influenced by the intervention in order to detect whether the outcomes reached the control group values.

### Results

There was no between-group difference for age, gender, height and weight (Table 4). One subject was removed from TBS group for technical issues. Scores from the initial clinical evaluation could not predict group changes in APA and TMS outcomes. Data are presented below for cTrA/IO and iTrA/IO activation during the postural task, for the corticomotor excitability, SICI and for pain self-assessment. No adverse effect was reported after TBS and TMS.

### Surface EMG Patterns During the Postural Task

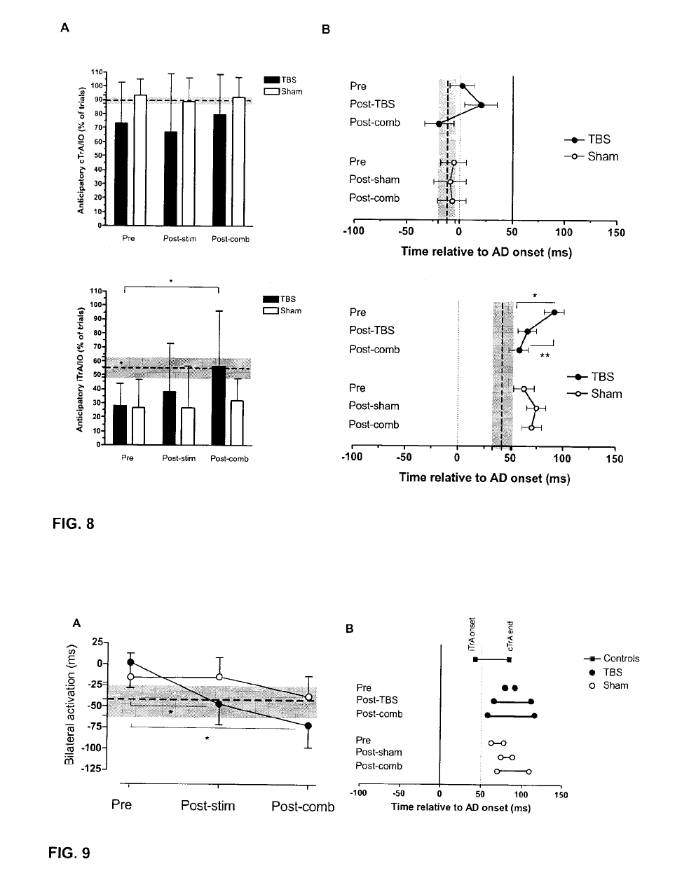

The two-way ANOVA_{RM} did not show any effect of the stimulation or combination on the percentage of trials with cTrA/IO anticipatory activation (F_{2,20}=0,39, p=0,685, FIG. 2A upper graph) and showed only a marginal GROUP X TIME interaction for cTrA/IO onset (F_{2,20}=2,78, p=0,086, FIG. 2B upper graph). For iTrA/IO anticipatory activation (FIG. 8A lower graph), the planned comparisons showed a 100,24% increase for TBS group at post-combination (mean=56,67% of trials, SD=39,89%) compared to pre-stimulation (mean=28,30% of trials, SD=16,02%; p=0,035) and to the non significant 20,6% increase in the Sham group. ANOVA_{RM} also detected a strong GROUP X TIME interaction for iTrA/10 onset (F_{2,20}=6,41, p=0,007, FIG. 8B lower graph) with a 36,83% improvement (earlier onset) for TBS group at post-combination (mean=57,39 ms, SEM=9,2 ms) compared to pre-stimulation (mean=90,85 ms, SEM=9,8 ms, p=0,01) and to the non significant 12,24% worsening (later onset) in the Sham group. Note that iTrA/10 onset was also improved after TBS alone (mean=65,10 ms, SEM=9,0 ms) with respect to pre-stimulation (p=0,004). The planned comparisons of the one-way ANOVA with GROUP (TBS, Sham, Control) showed that iTrA/IO onset was first significantly later in TBS group than in Control (p=0,008), and reached normal values at post-stimulation (p=0,122) and at post-combination (p=0,332). CTrA/IO and iTrA variables were not different between TBS and Sham groups at pre-stimulation (p>0,05).

The planned comparisons of the two-way ANOVA_{RM} applied on the duration of bilateral TrA/IO activation (FIG. 9A) showed an increase in TBS group at post-stimulation (mean=−46,4 ms, SEM=24,7 ms) compared to pre-stimulation (mean=2,10 ms, SEM=29,8 ms, p=0,01) and a further increase at post-combination (mean=−71,00 ms, SEM=26,7 ms, p=0,03). In other words, the bilateral activation was 73,1-ms improved in TBS group at post-combination and only 22,5-ms increased in the Sham group. FIG. 9B illustrates that in TBS group, the bilateral activation improvement was due to earlier activation of iTrA/10. The planned comparisons of the one-way ANOVA with GROUP (TBS, Sham, Control) showed that bilateral activation was first marginally shorter in TBS group than in Control (p=0,067), and reached normal values at post-stimulation (p=0,881) and at post-combination (p=0,61). It was not different between TBS and Sham groups at pre-stimulation (p>0,05).

### Corticomotor Excitability and SICI

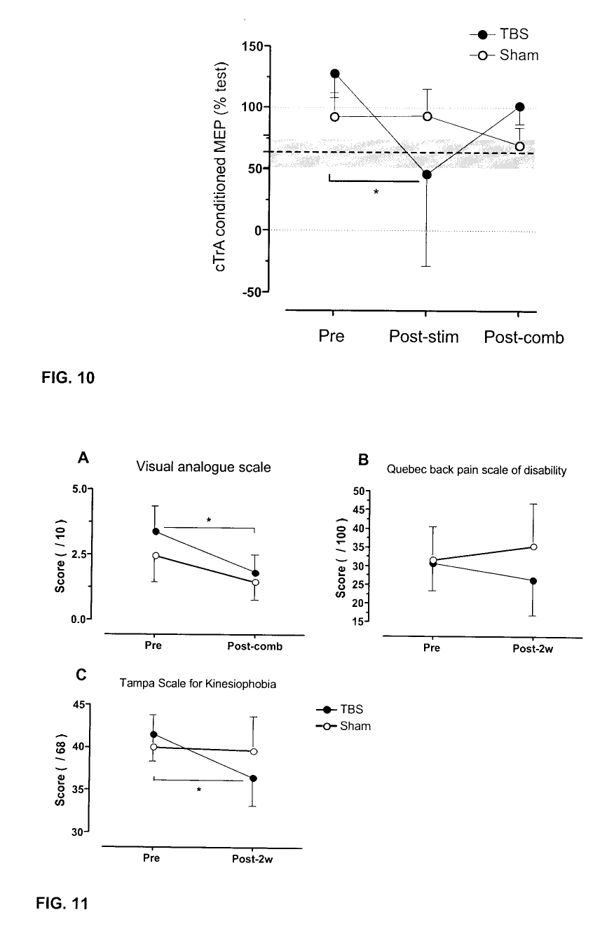

No effect of the stimulation or combination was detected for AMT and test MEP amplitudes of cTrA/IO. However, a strong GROUP X TIME interaction was detected for the conditioned MEP (F_{2,14}=5,06, p=0,022) with a significant decrease of conditioned MEP (SICI increase) in TBS group at post-stimulation (mean=45,90% of test MEP, SD=74,1%) compared to pre-stimulation (mean=128,10%, SD=20,30%, p=0,01, FIG. 10). There was however no change at post-combination compared to pre-stimulation and at any time for Sham group (p>0,05). The planned comparisons of the one-way ANOVA with GROUP (TBS, Sham, Control) showed that SICI was first missing in TBS group compared to Control (p=0,035), and reached values not different from Control group at post-stimulation (p=0,41) and at post-combination (p=0,135). SICI was not different between TBS and Sham groups at pre-stimulation (p>0,05).

### Pain Scores

A main effect of TIME on pain was detected (F_{1,10}=8,47; p=0,01) without difference between groups. However, the planned comparisons highlighted a significant decrease of pain in TBS group directly after the combination, as tested by VAS scale (mean=1,81/10, SEM=0,7) compared to pre-stimulation (mean=3,36/10, SEM=1,0, p=0,01, FIG. 11A) and to a marginal improvement in the Sham group (p=0,11). QBP scores (functional disability) was 4,4-point improved in TBS group two weeks after experiment (mean=26,1/100, SEM=9,5) without reaching significance when compared to pre-stimulation (mean=30,6/100, SEM=7,4, p=0,19, FIG. 11B). QBP scores were 3,8-point worsened in Sham group. TSK scores (kinesiophobia) were 5-point improved in TBS group two weeks after experiment (mean=36,4/68, SEM=3,3) as compared to pre-stimulation (mean=41,4/68, SEM=3,2, p=0,026, FIG. 5C) and to 0,4-point improvement in Sham group. Pain scores were not different between TBS and Sham groups at pre-stimulation (p>0,05).

### Discussion

This study presented for the first time that combining peripheral TBS and motor training of deep abdominal muscles influenced M1 function and improved motor control in CLBP subjects in a single session. This is discussed in terms of dynamic brain plasticity for motor programming and learning.

### Effects of Peripheral TBS on SICI

SICI is missing in chronic pain (Lefaucheur et al., 2006; Mhalla et al., 2010; Schwenkreis et al., 2003), precisely in CLBP (Masse-Alarie et al., submitted) and its restoration following M1 repetitive stimulation in pain is already reported (Lefaucheur et al., 2006; Mhalla et al., 2011). Our data on SICI reactivation by peripheral TBS over abdominal muscles in CLBP subjects are original. The mechanisms involved in such improvements remain speculative and may be related to CLBP condition itself. Indeed, sensory reafferences to S1 areas can be altered in CLBP by pain and posture (Masse-Alarie and Schneider, 2011) leading to maladaptation of sensorimotor areas resulting in proprioception alteration (O'Sullivan et al., 2003), tactile acuity extinction and disruption of body image (Moseley, 2008). FMRI studies showed a decrease in thalamus gray matter (Apkarian et al., 2004) and a medial shift in S1 (Flor et al., 1997) which relay information to M1 circuits. That is, S1-to-M1 transfer of information depends on the integrity of M1 intracortical connections (Kaneko et al., 1994). Thus, a missing SICI in CLBP subjects may reflect alteration of peripheral afferents or/and impaired reorganization of sensorimotor areas. That is, TBS of cTrA/10 led to muscle contraction either by direct stimulation of muscle fibers or via a preferential depolarization of axonal terminals (alpha-motoneurons) in the nerve (Struppler et al., 2003; Struppler et al., 2007). This indirect (contraction-related) generation of proprioceptive information to S1 is thought to be of great significance for brain in the induction of sensory-driven plasticity (Struppler et al., 2007). Peripheral TBS may have generated a sensory-driven activity in M1 circuits, thus compensating the pain-related impairment of sensory inputs integration and transiently reactivating SICI mechanisms.

### Effects of Combining Peripheral TBS and Training on SICI

The combination of peripheral TBS with training decreased SICI that was previously reactivated by TBS alone (FIG. 10). An explanation may specifically concern the underlying mechanisms of the learning task itself (motor training for isolating TrA/IO contraction) which was different from simply maintaining TrA/IO at 15% MVC. Indeed, during TMS testing at 15% MVC, participants had to draw-in their belly and reach visual feedback of EMG background with no other consign. During motor learning task, subjects received many verbal cues to succeed in isolating TrA/IO contraction and visual feedback was used to avoid EO cocontraction. Motor training was thus more complex, it required sustained attention and may have induced dynamic use-dependent plasticity in M1 circuits. In line, (Tsao et al., 2010) recently reported that TrA motor training normalized the shift of TrA M1 area in LBP subjects. However, they did not test SICI and motor training was conducted over 2 weeks, thus inducing cerebral changes different than those likely obtained in our single-session training study. In fact, SICI decrease after motor learning was reported only in distal muscles (Liepert et al., 1998; Smyth et al., 2010). Our study is the first to report SICI release from M1 circuits during motor training of abdominal muscles, at least in CLBP subjects. The underlying role of SICI release in learning is supported by a reduced use-dependent plasticity under pharmacological increase of SICI (Butefisch et al., 2000), thus suggesting that mechanisms of long-term potentiation (LTP) favor learning (Ziemann et al., 2004). Functionally, SICI release may reflect disinhibition of M1 horizontal connections thus increase of synaptic efficacy and recruitment of more corticospinal cells during motor training (Pascual-Leone et al., 1995). Our results in cTrA/IO muscle thus suggest that learning abdominal motor tasks shares similar M1 mechanisms with distal tasks.

Metaplasticity of M1 circuits (long-term depression-LTD following sustained activity and LTP following depressed activity, (Bienenstock et al., 1982)) may have influenced our SICI data because of the preactivated state of TrA/IO. However, such metaplasticity was documented only after repetitive magnetic stimulation of brain (Gentner et al., 2008), and not for peripheral TBS and SICI testing. Further studies should address this issue.

### Relevance of the Approach

The rationale behind combining peripheral TBS and motor training leans on the similar mechanisms of brain plasticity shared by the two interventions (Bolognini et al., 2009). Indeed, motor training and magnetic stimulation both activate N-methyl-D-aspartate and GABA receptors that are respectively involved in facilitation and inhibition at the basis of LTP/LTD induction in M1 circuits (Butefisch et al., 2000). Therefore, in our study, the use of TBS of cTrA/10 muscle seemed to prime M1 synaptic circuits before training, as revealed by the reactivation of the missing SICI after TBS alone (FIG. 4). SICI reactivation by TBS just prior to training may have in turn favored SICI release during motor training. Peripheral TBS prior to motor training of cTrA/IO could have thus provided the dynamic plastic conditions in M1 required for motor learning. This is also supported by the absence of effects in the Sham group.

### Influence on Postural Adjustments

Our study did not detect cTrA/IO delay in CLPB subjects but a delay in iTrA/IO, conversely to studies using fine-wire intramuscular recordings (Hodges and Richardson, 1996, 1998, 1999; Tsao et al., 2008b, 2010; Tsao and Hodges, 2007) but in line with other surface electrodes studies (Marshall and Murphy, 2003; Silfies et al., 2009). Anecdotally, further investigations should determine why surface vs. fine-wire intramuscular electrodes did not detect similar deficits in LBP population and whether the different etiology (recurrent vs. chronic LBP) and age of subjects in the different studies influenced the results.

Because iTrA/IO was not the stimulated side, the improvement of iTrA/IO anticipation was unexpected. The effect of cTrA/IO stimulation on iTrA/IO outcomes may result from the bilateral control of axial muscles by M1 (Carr et al., 1994; Strutton et al., 2004; Tsao et al., 2008a; Tunstill et al., 2001). Indeed, peripheral TBS on one side might have generated proprioceptive flows towards the contralateral M1, whose changes (SICI reactivation for example) could have influenced in turn both sides via bilateral descending pathways. The effect may be observed only in the muscle (iTrA/IO) with a delay detectable by the surface electrodes. Another explanation could be the influence of peripheral TBS at the spinal level; however, the unchanged test MEP amplitudes (reflecting corticospinal excitability) whereas SICI was restored, supports a dynamic plasticity at M1 level, SICI being of pure cortical origin (Di Lazzaro et al., 1998). Further studies should however thoroughly test the influence on the spinal circuits of abdominal muscles.

One another important finding was that combining training with peripheral TBS provided larger improvement of TrA/IO bilateral activation than sham combination, i.e. with activation overlap in TBS group having become similar to overlap in controls. Even if TrA activation is asymmetric between sides for a unilateral fast focal limb movement (Allison et al., 2008), the bilateral activation is anticipatory in most healthy subjects and is missing in most CLBP subjects (Massé-Alarie et al., submitted). Our results showed that iTrA/IO activation nearly reached anticipatory criteria (within 50 ms after AD onset) under combination thus supporting an adjuvant effect of peripheral TBS. Such improvements of temporal and spatial interactions between both TrA/10 during APA may reflect a better motor programming. The impact may be significant for rehabilitation because TBS-related improvement of TrA/10 bilateral activation may contribute to better control spine (Barker et al., 2004; Barker et al., 2006).

### Methodological Considerations

The absence of significant APA improvement when motor training was combined with sham stimulation is contrasted to peripheral TBS that improved the outcomes. The surface electrodes could not however be a confounding factor since the same electrodes were used in both groups of subjects for cTrA/IO and iTrA/IO. Also, we used a visual feedback of surface EMG recordings to monitor that EO was not activated during motor training. Surface recordings disabled discrimination between TrA and IO activation and IO co-contraction cannot be excluded. However, training TrA the way we did could be convenient clinically when real-time ultrasound device (feedback for TrA contraction alone during exercise) is not accessible. Since decrease in pain was not group-dependent (FIG. 5), it is not excluded that sham stimulation or manual therapy itself provided a single-session relief. Pain relief for TBS group could be attributed to peripheral neurostimulation itself as already reported for repetitive magnetic stimulation of M1 in chronic pain conditions (Lefaucheur et al., 2006; Lefaucheur et al., 2001; Mhalla et al., 2011). The decrease of TSK scores self-assessed 2 weeks after the experiment was surprising because a single-session training was supervised and no advice was given for fear-avoidance belief. This could be explained by the already reported relation between disability and kinesiophobia (Vlaeyen et al., 1995) though a slight decrease in disability. Altogether, these findings will have to be reproduced in larger samples of participants and over several sessions.

### Conclusions

This study provided the first evidence of the relevance of peripheral TBS as an adjuvant to motor training of TrA/IO muscles in individuals with CLBP. Spine control relying on the interaction of subsystems each involved in a specific component of spine stability (Panjabi, 1992a, b), this approach should be tested on other trunk and pelvis muscles whose training is relevant in CLBP rehabilitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All patents, patent applications and publications mentioned in this specification are herein incorporated by reference to the same extent as if each independent patent, patent application, or publication was specifically and individually indicated to be incorporated by reference.

### REFERENCES

1. Ziemann U, Paulus W, Nitsche M A et al. Consensus: Motor cortex plasticity protocols. Brain Stimul. 2008; 1(3):164-182.
2. Rothkegel H, Sommer M, Paulus W. Breaks during 5 Hz rTMS are essential for facilitatory after effects. Clinical Neurophysiology 2010; 121:426-430.
3. Siebner H R, Rothwell J. Transcranial magnetic stimulation: new insights into representational cortical plasticity. Exp Brain Res. 2003; 148(1):1-16.
4. Suppa A, Ortu E, Zafar N et al. Theta burst stimulation induces after-effects on contralateral primary motor cortex excitability in humans. J. Physiol. 2008; 586(18):4489-4500.
5. Màlly J, Dinya E. Recovery of motor disability and spasticity in post-stroke after repetitive transcranial magnetic stimulation (rTMS). Brain Research Bulletin 2008; 76:388-395.
6. Froc D J, Chapman C A, Trepel C et al. Long-term depression and depotentiation in the sensorimotor cortex of the freely moving rat. The Journal of Neuroscience 2000; 20(1):438-445.
7. Maeda F, Keenan J P, Tormos J M et al. Modulation of corticospinal excitability by repetitive transcranial magnetic stimulation. Clinical Neurophysiology 2000; 111:800-805.
8. Huang Y Z, Rothwell J C. The effect of short-duration bursts of high-frequency, low-intensity transcranial magnetic stimulation on the human motor cortex. Clin Neurophysiol. 2004; 115(5):1069-1075.
9. Huang Y Z, Edwards M J, Rounis E et al. Theta burst stimulation of the human motor cortex. Neuron. 2005; 45:201-206.
10. Talelli P, Greenwood R J, Rothwell J C. Exploring Theta Burst Stimulation as an intervention to improve motor recovery in chronic stroke. Clinical Neurophysiology 2007; 118:333-342.
11. Huang Y Z, Rothwell J C, Edwards M J et al. Effect of physiological activity on an NMDA-dependent form of cortical plasticity in human. Cerebral Cortex 2008; 18:563-570.
12. Huang Y Z, Sommer M, Thickbroom G et al. Consensus: New methodologies for brain stimulation. Brain Stimul. 2009; 2(1):2-13.
13. Gentner R, Wanker K, Reinsberger C et al. Depression of human corticospinal excitability induced by magnetic theta-burst stimulation: evidence of rapid polarity-reversing metaplasticity. Cerebral Cortex 2008; 18:2046-2053.
14. Gamboa O L, Antal A, Laczo B et al. Impact of repetitive theta burst stimulation on motor cortex excitability. Brain Stimulation. Forthcoming 2010.
15. Siebner H R. A primer on priming the human motor cortex. Clin Neurophysiol. 2010; 121(4):461-463.
16. Rossi S, Hallett M, Rossini P M et al. Safety, ethical considerations, and application guidelines for the use of transcranial magnetic stimulation in clinical practice and research. Clin Neurophysiol. 2009; 120(12):2008-2039.
17. Struppler A, Havel P, Müller-Barna P. Facilitation of skilled finger movements by repetitive peripheral magnetic stimulation (RPMS)—a new approach in central paresis. NeuroRehabilitation 2003; 18:69-82.
18. Nielsen J F, Sinkjaer T, Jakobsen J. Treatment of spasticity with repetitive magnetic stimulation; a double-blind placebo-controlled study. Mult Scler. 1996; 2(5):227-232.
19. Nielsen J F, Sinkjaer T. Long-lasting depression of soleus motoneurons excitability following repetitive magnetic stimuli of the spinal cord in multiple sclerosis patients. Mult Scler. 1997; 3(1):18-30.
20. Krause P, Straube A. Repetitive magnetic and functional electrical stimulation reduce spastic tone increase in patients with spinal cord injury. Suppl Clin Neurophysiol. 2003; 56:220-225.
21. Krause P, Edrich T, Straube A. Lumbar repetitive magnetic stimulation reduces spastic tone increase of the lower limbs. Spinal Cord 2004; 42:67-72.
22. Havel P, Struppler A. First steps in functional magnetic stimulation (FMS)-movements of forearm and fingers induced by closed-loop controlled FMS. Acta Physiol Pharmacol Bulg. 2001; 26(3):185-188.
23. Gamboa O L, Antal A, Moliadze V et al. Simply longer is not better: reversal of theta burst after-effect with prolonged stimulation. Exp Brain Res. 2010; 204:181-187.
24. Oldfield R C. The assessment and analysis of handedness: The Edinburgh Inventory. Neuropsychologia 1971; 9(1):97-113.
25. Loder R T, Browne R, Bellflower J et al. Angular measurement error due to different measuring devices. J Pediatr Orthop. 2007; 27:338-346.
26. Young R R, Wiegner A W. Spasticity. Clin Orthop Relat Res. 1987; 219:50-62.
27. Lamontagne A, Malouin F, Richards C L, et al. Evaluation of Reflex- and Nonreflex- induced muscle resistance to stretch in adults with spinal cord injury using hand-held and isokinetic dynamometry. Physical Therapy 1998; 78(9):964-978.
28. Howell D C. Méthodes statistiques en sciences humaines. Paris: De Boeck Universite; 1998.
29. Mori F, Koch G, Foti C et al. The use of repetitive transcranial magnetic stimulation (rTMS) for the treatment of spasticity. Progress in Brain Research 2009; 175:429-439.
30. Nowak D A, Grefkes C, Ameli M et al. Interhemispheric competition after stroke: Brain stimulation to enhance recovery of function of the affected hand. Neurorehabil Neural Repair 2009; 23(7):641-656.
31. Ward N S, Brown M M, Thompson A J et al. Longitudinal changes in cerebral response to proprioceptive input in individual patients after stroke: An fMRI study. Neurorehabil Neural Repair 2006; 20(3):398-405.
32. Wolters A, Schmidt A, Schramm A et al. Timing-dependent plasticity in human primary somatosensory cortex. J. Physiol. 2005; 565(3):1039-1052.
33. Nielsen J B, Crone C, Hultborn H. The spinal pathophysiology of spasticity—from a basic science point of view. Acta Physiol (Oxf). 2007; 189(2):171-180.
34. Mori F, CodecàC, Kusayanagi H et al. Effect of intermittent theta burst stimulation on spasticity in patient with multiple sclerosis. European Journal of Neurology 2010; 17:295-300.
35. Page S J, Gater D R, Bach-Y-Rita P. Reconsidering the motor recovery plateau in stroke rehabilitation. Arch Phys Med Rehabil. 2004; 85(8):1377-1381.
Allison, G. T., Morris, S. L., and Lay, B. (2008). Feedforward responses of transversus abdominis are directionally specific and act asymmetrically: implications for core stability theories. J Orthop Sports Phys Ther 38, 228-237.
Apkarian, A. V., Sosa, Y., Sonty, S., Levy, R. M., Harden, R. N., Parrish, T. B., and Gitelman, D. R. (2004). Chronic back pain is associated with decreased prefrontal and thalamic gray matter density. J Neurosci 24, 10410-10415.
Aruin, A. S., and Latash, M. L. (1995). Directional specificity of postural muscles in feed-forward postural reactions during fast voluntary arm movements. Experimental Brain Research 103, 323-332.
Barker, P. J., Briggs, C. A., and Bogeski, G. (2004). Tensile transmission across the lumbar fasciae in unembalmed cadavers: effects of tension to various muscular attachments. Spine (Phila Pa 1976) 29, 129-138.
Barker, P. J., Guggenheimer, K. T., Grkovic, I., Briggs, C. A., Jones, D. C., Thomas, C. D., and Hodges, P. W. (2006). Effects of tensioning the lumbar fasciae on segmental stiffness during flexion and extension: Young Investigator Award winner. Spine (Phila Pa 1976) 31, 397-405.
Bergmark, A. (1989). Stability of the lumbar spine. A study in mechanical engineering. Acta Orthop Scand Suppl 230, 1-54.
Bienenstock, E. L., Cooper, L. N., and Munro, P. W. (1982). Theory for the development of neuron selectivity: orientation specificity and binocular interaction in visual cortex. J Neurosci 2, 32-48.
Bolognini, N., Pascual-Leone, A., and Fregni, F. (2009). Using non-invasive brain stimulation to augment motor training-induced plasticity. Journal of NeuroEngineering and Rehabilitation 6, 8.

Butefisch, C., Davis, B., Wise, S., Sawaki, L., Kopylev, L., Classen, J., and Cohen, L. (2000). Mechanisms of use-dependent plasticity in the human motor cortex. Proc Natl Acad Sci USA 97, 3661-3665.
Carr, L. J., Harrison, L. M., and Stephens, J. A. (1994). Evidence for bilateral innervation of certain homologous motoneurone pools in man. J Physiol 475, 217-227.
Di Lazzaro, V., Restuccia, D., Oliviero, A., Profice, P., Ferrara, L., Insola, A., Mazzone, P., Toneli, P., and Rothwell, J. C. (1998). Magnetic transcranial stimulation at intensities below active motor threshold activates intracortical inhibitory circuits. Exp Brain Res 119, 265-268.
Flor, H., Braun, C., Elbert, T., and Birbaumer, N. (1997). Extensive reorganization of primary somatosensory cortex in chronic back pain patients. Neuroscience Letters 224, 5-8.
French, D. J., Roach, P. J., and Mayes, S. (2002). Peur du mouvement chez des accidentes du travail: L′Échelle de Kinesiophobie de Tampa (EKT). [Fear of movement in injured workers: The Tampa Scale of Kinesiophobia.]. Canadian Journal of Behavioural Science/Revue canadienne des sciences du comportement 34, 28-33.
Gagné, M., and Schneider, C. (2008). Dynamic influence of wrist flexion and extension on the intracortical inhibition of the first dorsal interosseus muscle during precision grip. Brain Research 1195, 77-88.
Gentner, R., Wankerl, K., Reinsberger, C., Zeller, D., and Classen, J. (2008). Depression of human corticospinal excitability induced by magnetic theta-burst stimulation: evidence of rapid polarity-reversing metaplasticity. Cereb Cortex 18, 2046-2053.
Hayden, J., van Tulder, M. W., Malmivaara, A., and Koes, B. W. (2010). Exercise therapy for treatment of non-specific low back pain (Review). In Cochrane collaboration (John Wiley & Sons, Ltd), p. 99.
Hicks, G. E., Fritz, J. M., Delitto, A., and McGill, S. M. (2005). Preliminary development of a clinical prediction rule for determining which patients with low back pain will respond to a stabilization exercise program. Arch Phys Med Rehabil 86, 1753-1762.
Hodges, P. W. (2011). Pain and motor control: From the laboratory to rehabilitation. J Electromyogr Kinesiol 21, 220-228.
Hodges, P. W., and Bui, B. H. (1996). A comparison of computer-based methods for the determination of onset of muscle contraction using electromyography. Electroencephalogr Clin Neurophysiol 101, 511-519.
Hodges, P. W., Ferreira, H. F., and Ferreira, M. L. (2009). Lumbar spine: Treatment of instability and disorders of movement control. In Pathology and intervention in musculoskeletal rehabilitation., D. J. Magee, J. E. Zachazewski, and W. S. Quillen, eds. (St. Louis: Saunders Elsevier), pp. 389-425.
Hodges, P. W., and Richardson, C. A. (1996). Inefficient muscular stabilization of the lumbar spine associated with low back pain. A motor control evaluation of transversus abdominis. Spine (Phila Pa 1976) 21, 2640-2650.
Hodges, P. W., and Richardson, C. A. (1998). Delayed postural contraction of transversus abdominis in low back pain associated with movement of the lower limb. J Spinal Disord 11, 46-56.
Hodges, P. W., and Richardson, C. A. (1999). Altered trunk muscle recruitment in people with low back pain with upper limb movement at different speeds. Arch Phys Med Rehabil 80, 1005-1012.
Huang, Y., Edwards, M., Rounis, E., Bhatia, K., and Rothwell, J. (2005). Theta burst stimulation of the human motor cortex. Neuron 45, 201-206.
Kaneko, T., Caria, M. A., and Asanuma, H. (1994). Information processing within the motor cortex. II. Intracortical connections between neurons receiving somatosensory cortical input and motor output neurons of the cortex. The Journal of Comparative Neurology 345, 172-184.
Kopec, J. A., Esdaile, J. M., Abrahamowicz, M., Abenhaim, L., Wood-Dauphinee, S., Lamping, D. L., and Williams, J. I. (1995). The Quebec Back Pain Disability Scale. Measurement properties. Spine (Phila Pa 1976) 20, 341-352.
Kujirai, T., Caramia, M. D., Rothwell, J. C., Day, B. L., Thompson, P. D., Ferbert, A., Wroe, S., Asselman, P., and Marsden, C. D. (1993). Corticocortical inhibition in human motor cortex. The Journal of Physiology 471 501-519
Lefaucheur, J. P., Drouot, X., Menard-Lefaucheur, I., Keravel, Y., and Nguyen, J. P. (2006). Motor cortex rTMS restores defective intracortical inhibition in chronic neuropathic pain. Neurology 67, 1568-1574.
Lefaucheur, J. P., Drouot, X., and Nguyen, J. P. (2001). Interventional neurophysiology for pain control: duration of pain relief following repetitive transcranial magnetic stimulation of the motor cortex. Neurophysiol Clin 31, 247-252.
Liepert, J., Classen, J., Cohen, L. G., and Hallett, M. (1998). Task-dependent changes of intracortical inhibition. Experimental Brain Research 118, 421-426.
Maccabee, P. J., Amassian, V. E., Cracco, R. Q., and Cadwell, J. A. (1988). An analysis of peripheral motor nerve stimulation in humans using the magnetic coil. Electroencephalogr Clin Neurophysiol 70, 524-533.
Maeda, F., Keenan, J., Tormos, J., Topka, H., and Pascual-Leone, A. (2000). Interindividual variability of the modulatory effects of repetitive transcranial magnetic stimulation on cortical excitability. Exp Brain Res 133, 425-430.
Marshall, P., and Murphy, B. (2003). The validity and reliability of surface EMG to assess the neuromuscular response of the abdominal muscles to rapid limb movement. Journal of Electromyography and Kinesiology 13, 477-489.
Masse-Alarie, H., and Schneider, C. (2011). [Cerebral reorganization in chronic low back pain and neurostimulation to improve motor control]. Neurophysiol Clin 41, 51-60.
Mhalla, A., Baudic, S., de Andrade, D. C., Gautron, M., Perrot, S., Teixeira, M. J., Attal, N., and

Bouhassira, D. (2011). Long-term maintenance of the analgesic effects of transcranial magnetic stimulation in fibromyalgia. Pain 152, 1478-1485.
Mhalla, A., de Andrade, D. C., Baudic, S., Perrot, S., and Bouhassira, D. (2010). Alteration of cortical excitability in patients with fibromyalgia. Pain 149, 495-500.
Moseley, G. L. (2008). I can't find it! Distorted body image and tactile dysfunction in patients with chronic back pain. Pain 140, 239-243.
Ng, J. K., Kippers, V., Parnianpour, M., and Richardson, C. A. (2002). EMG activity normalization for trunk muscles in patients with and without back pain. Med Sci Sports Exerc 34, 1082-1086.
O'Sullivan, P. B., Burnett, A., Floyd, A. N., Gadsdon, K., Logiudice, J., Miller, D., and Quirke, H. (2003). Lumbar repositioning deficit in a specific low back pain population. Spine (Phila Pa 1976) 28, 1074-1079.
Ortu, E., Deriu, F., Suppa, A., Tolu, E., and Rothwell, J. C. (2008). Effects of volitional contraction on intracortical inhibition and facilitation in the human motor cortex. The Journal of Physiology 586, 5147-5159.
Panjabi, M. M. (1992a). The stabilizing system of the spine. Part I. Function, dysfunction, adaptation, and enhancement. J Spinal Disord 5, 383-389; discussion 397.
Panjabi, M. M. (1992b). The stabilizing system of the spine. Part II. Neutral zone and instability hypothesis. J Spinal Disord 5, 390-396; discussion 397.
Pascual-Leone, A., Nguyet, D., Cohen, L., Brasil-Neto, J., Cammarota, A., and Hallett, M. (1995). Modulation of muscle responses evoked by transcranial magnetic stimulation during the acquisition of new fine motor skills. J Neurophysiol 74, 1037-1045.
Richardson, C. A., Hides, J. A., Wilson, S., Stanton, W., and Snijders, C. J. (2004). Lumbo-pelvic joint protection against antigravity forces: motor control and segmental stiffness assessed with magnetic resonance imaging. J Gravit Physiol 11, P119-122.
Ridding, M. C., and Rothwell, J. C. (1999). Afferent input and cortical organisation: a study with magnetic stimulation. Exp Brain Res 126, 536-544.
Rossi, S., Hallett, M., Rossini, P. M., and Pascual-Leone, A. (2009). Safety, ethical considerations, and application guidelines for the use of transcranial magnetic stimulation in clinical practice and research. Clin Neurophysiol 120, 2008-2039.
Sakai, K., Ugawa, Y., Terao, Y., Hanajima, R., Furubayashi, T., and Kanazawa, I. (1997). Preferential activation of different I waves by transcranial magnetic stimulation with a figure-of-eight-shaped coil. Exp Brain Res 113, 24-32.
Schwenkreis, P., Janssen, F., Rommel, 0., Pleger, B., Volker, B., Hosbach, I., Dertwinkel, R., Maier, C., and Tegenthoff, M. (2003). Bilateral motor cortex disinhibition in complex regional pain syndrome (CRPS) type I of the hand. Neurology 61, 515-519.

Silfies, S. P., Mehta, R., Smith, S. S., and Karduna, A. R. (2009). Differences in Feedforward Trunk Muscle Activity in Subgroups of Patients With Mechanical Low Back Pain. Archives of Physical Medicine and Rehabilitation 90, 1159-1169. Smyth, C., Summers, J. J., and Garry, M. I. (2010). Differences in motor learning success are associated with differences in M1 excitability. Human Movement Science 29, 618-630.

Struppler, A., Angerer, B., and Havel, P. (2003). Modulation of sensorimotor performances and cognition abilities induced by RPMS: clinical and experimental investigations. Suppl Clin Neurophysiol 56, 358-367.

Struppler, A., Binkofski, F., Angerer, B., Bernhardt, M., Spiegel, S., Drzezga, A., and Bartenstein, P. (2007). A fronto-parietal network is mediating improvement of motor function related to repetitive peripheral magnetic stimulation: A PET-H2O15 study. NeuroImage Cortical Control of Higher Motor Cognition 36, T174-T186.
Strutton, P. H., Beith, I. D., Theodorou, S., Catley, M., McGregor, A. H., and Davey, N. J. (2004). Corticospinal activation of internal oblique muscles has a strong ipsilateral component and can be lateralised in man. In Experimental Brain Research (Springer Berlin/Heidelberg), pp. 474-479.
Tsao, H., Galea, M. P., and Hodges, P. W. (2008a). Concurrent excitation of the opposite motor cortex during transcranial magnetic stimulation to activate the abdominal muscles. J Neurosci Methods 171, 132-139.
Tsao, H., Galea, M. P., and Hodges, P. W. (2008b). Reorganization of the motor cortex is associated with postural control deficits in recurrent low back pain. Brain 131, 2161-2171.
Tsao, H., Galea, M. P., and Hodges, P. W. (2010). Driving plasticity in the motor cortex in recurrent low back pain. Eur J Pain 14, 832-839.
Tsao, H., and Hodges, P. W. (2007). Immediate changes in feedforward postural adjustments following voluntary motor training. Exp Brain Res 181, 537-546.
Tunstill, S., Wynn-Davies, A., Nowicky, A., McGregor, A., and Davey, N. (2001). Corticospinal facilitation studied during voluntary contraction of human abdominal muscles Experimental Physiology 86 131-136
Urquhart, D. M., Hodges, P. W., Allen, T. J., and Story, I. H. (2005). Abdominal muscle recruitment during a range of voluntary exercises. Man Ther 10, 144-153.
Vlaeyen, J. W. S., Kole-Snijders, A. M. J., Rotteveel, A. M., Ruesink, R., and Heuts, P. H. T. G. (1995). The role of fear of movement/(re)injury in pain disability. Journal of Occupational Rehabilitation 5, 235-252.
Zhu, Y., and Starr, A. (1991). Magnetic stimulation of muscle evokes cerebral potentials. Muscle Nerve 14, 721-732.
Ziemann, U., Ilic, T. V., Pauli, C., Meintzschel, F., and Ruge, D. (2004). Learning modifies subsequent induction of long-term potentiation-like and long-term depression-like plasticity in human motor cortex. J Neurosci 24, 1666-1672.

1. A method for improving motor control impairment of a subject suffering therefrom, comprising the step of applying theta-burst stimulation (TBS) to a peripheral nerve or muscle of said subject.

2. The method according to claim 1, wherein said TBS is intermittent (iTBS), wherein said muscle is stimulated to improve motor control thereof.

3. The method of claim 2, wherein said iTBS is applied to a peripheral nerve of said muscle or an associated muscle thereof.

4. The method of claim 2, wherein said iTBS is applied directly to said muscle or an associated muscle thereof.

5. The method of claim 2, wherein said iTBS is applied to a peripheral nerve of an opposite muscle thereof.

6. The method of claim 2, wherein said iTBS is applied directly to an opposite muscle thereof.

7. The method of claim 1, wherein the subject is a human subject.

8. The method of claim 7, wherein said subject is selected from the group consisting of: a premature baby, or a subject having chronic back pain, peripheral muscle rigidity or peripheral muscle spasticity.

9. The method according to claim 8, wherein said motor-control impairment causes chronic lower back pain, wherein said muscle is stimulated to improve motor-control thereof.

10. The method according to claim 8, wherein said motor-control impairment is muscle rigidity, wherein said muscle is stimulated to decrease rigidity thereof.

11. The method of claim 10, wherein said subject having peripheral muscle rigidity is a subject having Parkinson's disease.

12. The method according to claim 8, for the treatment of muscular spasticity of a paretic subject comprising the steps of: applying theta-burst stimulation (TBS) to peripheral nerves of a paretic muscle of said subject.

13. The method of claim 12, comprising the steps of:
intermittently applying theta-burst stimulation to peripheral nerves of a first paretic muscle; and
continuously applying theta-burst stimulation to peripheral nerves of a second paretic muscle;
wherein said first and second paretic muscles act in connection with each other for achieving limb movement.

14. The method of claim 13, wherein the peripheral theta-burst stimulation (PTBS) is applied to a first paretic muscle and a second paretic muscle sequentially.

15. The method of claim 14, wherein the PTBS is applied sequentially as continuous TBS (cTBS) on a first paretic hyperactivated muscle and as intermittent TBS (iTBS) on a second paretic underactivated muscle.

16. The method of claim 15, wherein the first paretic muscle is stimulated by cTBS to decrease spasticity of the first muscle and the second paretic muscle is stimulated by iTBS to increase contraction of the second muscle.

17. The method of claim 12, wherein the subject is a human subject.

18. The method of claim 17, wherein the human subject is paretic and suffers neuronal loss because of stroke, aneurysm rupture, cerebral palsy or acquired brain injury (ABI).

19. The method of claim 1, wherein PTBS are delivered with 5-Hz bursts of three pulses at 50 Hz.

20. The method of claim 19, wherein cTBS₆₀₀ consists of a 40-sec train of uninterrupted PTBS (600 pulses) and iTBS₆₀₀ of a 2-sec train of PTBS repeated every 10 sec for a total of 190 sec (600 pulses).

21.-60. (canceled)
